(19) United States
(12) Patent Application Publication
(10) Pub. No.: US 2012/0283175 A1
(43) Pub. Date: Nov. 8, 2012

(54) ANTIBACTERIAL COMPOSITIONS

(75) Inventors: Phillip A. Patten, Portola Valley, CA (US); Eliana Saxon Armstrong, San Mateo, CA (US)

(73) Assignee: Achaogen, Inc., South San Francisco, CA (US)

(21) Appl. No.: 13/289,212

(22) Filed: Nov. 4, 2011

Related U.S. Application Data
(63) Continuation of application No. PCT/US2010/033910, filed on May 6, 2010.
(60) Provisional application No. 61/176,297, filed on May 7, 2009.

(51) Int. Cl. A61K 31/5375; A61K 31/7052; A61K 31/496; A61K 31/5377; A61P 31/04; A61K 31/546; A61K 31/7036; A61K 31/7048; A61K 31/407; A61K 38/14; A61K 31/7028

(52) U.S. Cl. 514/2.8; 514/3.1; 514/29; 514/254.11; 514/2.9; 514/236.8; 514/24; 514/202; 514/40; 514/27; 514/210.1; 514/238.2

(57) ABSTRACT

The present invention provides a methods and pharmaceutical compositions useful for treating bacterial infections in humans and animals which comprises administering to a human or animal in need thereof, a synergistic combination of an inhibitor of LpxC and second antibacterial agent.

### CROSS-REFERENCE TO RELATED APPLICATIONS

This application is a continuation of International PCT Patent Application No. PCT/US2010/033910, which was filed on May 6, 2010, now pending, which claims the benefit under 35 U.S.C. §119(e) of U.S. Provisional Patent Application No. 61/176,297 filed May 7, 2009. The foregoing applications are incorporated herein by reference in their entireties.

### STATEMENT OF GOVERNMENT INTEREST

This invention was made with government support under Contract No. HDTRA1-07-C-0079 awarded by the United States Department of Defense. The government has certain rights in this invention.

### BACKGROUND OF THE INVENTION

1. Field of the Invention

This invention pertains generally to treating infections caused by gram-negative bacteria and enhancing the activity of large antibacterial compounds such as vancomycin and rifampin. More specifically, the invention described herein pertains to treating gram-negative infections by inhibiting activity of UDP-3-O—(R-3-hydroxydecanoyl)-N-acetylglucosamine deacetylase (LpxC) in combination with administering a second antibacterial agent.

2. Description of the Related Art

Over the past several decades, the frequency of antimicrobial resistance and its association with serious infectious diseases have increased at alarming rates. The increasing prevalence of resistance among nosocomial pathogens is particularly disconcerting. Of the over 2 million nosocomial infections occurring each year in the United States, 50 to 60% are caused by antimicrobial-resistant strains of bacteria. This high rate of resistance increases the morbidity, mortality, and costs associated with nosocomial infections. In the United States, nosocomial infections are thought to contribute to or cause more than 77,000 deaths per year and cost approximately $5 to $10 billion annually. Among gram-positive organisms, the most important resistant pathogens are methicillin-(oxacillin-)resistant Staphylococcus aureus, β-lactam-resistant and multidrug-resistant pneumococci, and vancomycin-resistant enterococci. Important causes of gram-negative resistance include extended-spectrum β-lactamases (ESBLs) in Klebsiella pneumoniae, Escherichia coli, and Proteus mirabilis, high-level third-generation cephalosporin (Amp C) β-lactamase resistance among Enterobacter species and Citrobacter freundii, and multidrug-resistance genes observed in Pseudomonas aeruginosa, Acinetobacter, and Stenotrophomonas maltophilia (see Jones, R. N., “Resistance patterns among nosocomial pathogens: Trends over the past few years” Chest. 119 (Supp 2):397S-404S (2001)).

The problem of antibacterial resistance is compounded by the existence of bacterial strains resistant to multiple antibacterials. For example, Pseudomonas aeruginosa isolates resistant to fluoroquinolones are virtually all resistant to additional antibacterials (see Sahm, D. F. et al., “Evaluation of current activities of fluoroquinolones against gram-negative bacilli using centralized in vitro testing and electronic surveillance” Antimicrobial Agents and Chemotherapy 45:267-274 (2001)). Thus there is a need for new antibacterial regimens, particularly antibacterial regimens that minimize development of antibacterial resistance.

Most of the antibacterial discovery effort in the pharmaceutical industry is aimed at development of drugs effective against gram-positive bacteria. However, there is also a need for new gram-negative antibacterials. Gram-negative bacteria are in general more resistant to a larger number of antibacterials and chemotherapeutic agents than are gram-positive bacteria. A survey of recently reported antibacterials of natural origin showed that over 90% lacked activity against Escherichia coli, although they were active against gram-positive bacteria. The outer membrane of gram-negative bacteria contributes to this intrinsic resistance by acting as an efficient permeability barrier, because the narrow porin channels limit the penetration of hydrophilic solutes and the low fluidity of the lipopolysaccharide leaflet slows down the inward diffusion of lipophilic solutes. Young and Silver (J. Bacteriol. 173(12):3609-14 (1991)) demonstrated that an envA1 strain, having an altered outer membrane, is sensitive to a variety of large and hydrophobic antibacterials to which wild type E. coli is resistant. It is believed that the outer membrane normally excludes hydrophilic compounds larger than about 600 Da, the molecular weight cutoff of outer membrane porins, and relatively hydrophobic drugs. Young and Silver hypothesized that increasing the permeability of the outer membrane will render E. coli sensitive to a variety of large hydrophilic or hydrophobic antibacterial agents. Vaara, et al., (Antimicrobial Agents and Chemotherapy 37(11):2255-2260 (1993)) review a variety of outer membrane-defective mutants of E. coli and S. typhimurium that show greater susceptibility than the corresponding wild type strain to a variety of antibacterial agents.

The present invention provides synergistic combinations of antibacterial agents with LpxC inhibitors, which have intrinsic antibacterial properties as well the ability to improve permeability of the outer membrane of gram-negative bacteria to other antibacterial agents. The use of synergistic combinations of drugs could have many advantages over conventional single compound chemotherapy, including lowered side-effects of drugs due to lower doses used or shorter time of treatment, more rapid cure of infection shortening hospital stays, increasing spectrum of pathogens controlled, and decreasing incidence of development of resistance to antibiotics

### BRIEF SUMMARY OF THE INVENTION

One aspect of the invention provides pharmaceutical compositions comprising a synergistic combination of an antibacterial agent and an inhibitor of LpxC. In a preferred embodiment, the synergistic combination demonstrates in vivo synergy.

In one embodiment, the antibacterial agent is selected from the group consisting of vancomycin, linezolid, azithromycin, imipenem, teicoplanin, daptomycin, clindamycin, rifampin, cefotaxime, gentamicin, novobiocin, and telavancin. In a more specific embodiment, the antibacterial agent is vancomycin or rifampin.

In another embodiment, the LpxC inhibitor is a compound of formula (I):

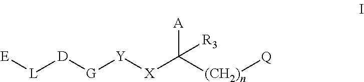

or a stereoisomer, pharmaceutically acceptable salt, ester, or prodrug thereof, as disclosed in greater detail below. In more specific embodiments, the LpxC inhibitor is selected from the group consisting of (R)—N-hydroxy-2-(4-methoxyphenyl)-4,5-dihydrooxazole-4-carboxamide (LpxCi-1); (S)-2-(3,4-dimethoxy-5-propylphenyl)-N-hydroxy-4,5-dihydrooxazole-4-carboxamide (LpxCi-2); N-((2S,3R)-3-hydroxy-1-(hydroxyamino)-1-oxobutan-2-yl)-4-((4-(morpholinomethyl)phenyl)ethynyl)benzamide (LpxCi-3); (S)—N-(3-amino-1-(hydroxyamino)-3-methyl-1-oxobutan-2-yl)-4-(cyclopropylbuta-1,3-diynyl)benzamide (LpxCi-4); (S,E)-N-(3-amino-1-(hydroxyamino)-3-methyl-1-oxobutan-2-yl)-4-(4-cyclopropylbut-3-en-1-ynyl)benzamide (LpxCi-5); and (S)-4-(cyclopropylbuta-1,3-diynyl)-N-(3-hydroxy-1-(hydroxyamino)-3-methyl-1-oxobutan-2-yl)benzamide (LpxCi-6).

In another embodiment, the LpxC inhibitor is selected from compounds having formula II-A, II-B or II-C:

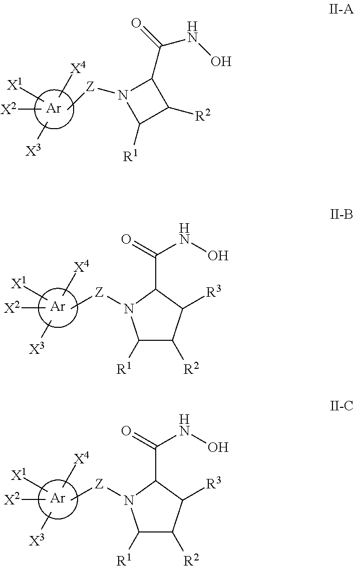

or a stereoisomer, pharmaceutically acceptable salt, ester, or prodrug thereof, as disclosed in greater detail below.

In yet another embodiment, the LpxC inhibitor is selected from compounds having formula III:

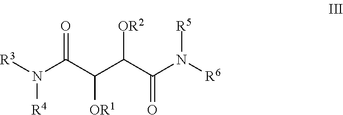

or a stereoisomer, pharmaceutically acceptable salt, ester, or prodrug thereof, as disclosed in greater detail below.

Another aspect of the invention provides methods for treating a patient with a gram-negative bacterial infection, comprising co-administering a synergistic amount, preferably an in vivo synergistic amount, of an antibacterial agent and an inhibitor of LpxC. In one embodiment, the antibacterial agent is selected from the group consisting of vancomycin, linezolid, azithromycin, imipenem, teicoplanin, daptomycin, clindamycin, rifampin, cefotaxime, gentamicin, novobiocin, and telavancin. In a more specific embodiment, the antibacterial agent is vancomycin or rifampin. In another embodiment, the LpxC inhibitor is a compound of formula I, II-A, II-B, II-C, or III, or a stereoisomer, pharmaceutically acceptable salt, ester, or prodrug thereof, as disclosed in greater detail below.

Yet another aspect of the invention provides methods of suppressing the emergence of resistance to an antibacterial agent, said method comprising co-administering a synergistic amount, preferably an in vivo synergistic amount, of the antibacterial agent and an inhibitor of LpxC. In one embodiment, the antibacterial agent is selected from the group consisting of vancomycin, linezolid, azithromycin, imipenem, teicoplanin, daptomycin, clindamycin, rifampin, cefotaxime, gentamicin, novobiocin, and telavancin. In a more specific embodiment, the antibacterial agent is vancomycin or rifampin. In another embodiment, the LpxC inhibitor is a compound of formula I, II-A, II-B, II-C, or III, or a stereoisomer, pharmaceutically acceptable salt, ester, or prodrug thereof, as disclosed in greater detail below.

### BRIEF DESCRIPTION OF THE DRAWINGS

FIG. 1 provides a copy of Table 3 from prior art reference Young et al., “Leakage of Periplasmic Enzymes from envA1 Strains of Escherichia coli,” J. Bacterial., Vol. 173, No. 12, pp. 3609-14 (1991).

FIG. 2 illustrates in vivo synergy of LpxCi-3 and vancomycin in bacterial strain ATCC43816.

FIG. 3 illustrates in vivo synergy of LpxCi-4 and vancomycin in bacterial strain ATCC43816.

FIG. 4 illustrates in vivo synergy of LpxCi-4 and rifampin in bacterial strain ATCC43816.

FIG. 5 illustrates in vivo synergy of LpxCi-4 and vancomycin in bacterial strain ATCC27853.

FIG. 6 illustrates in vivo synergy of LpxCi-6 and vancomycin in bacterial strain ATCC27853.

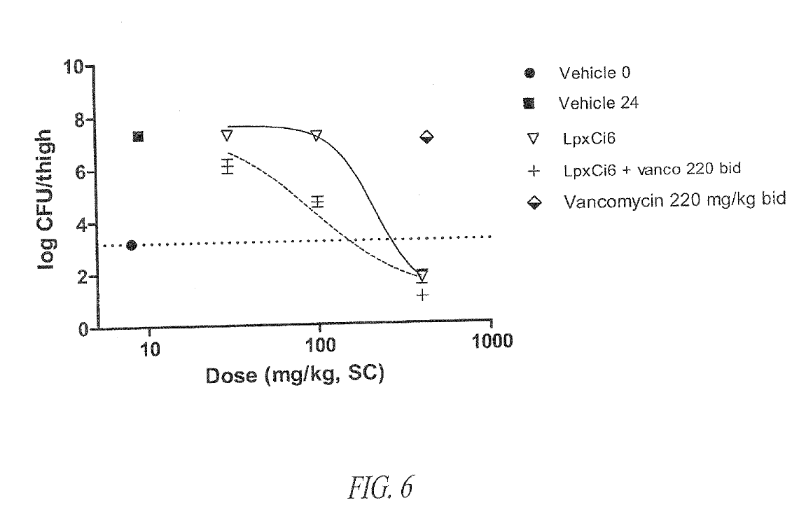

FIG. 7 illustrates in vivo synergy of LpxCi-4 and rifampin in bacterial strain ATCC27853.

FIG. 8 illustrates modest in vivo synergy of LpxCi-4 and erythromycin in bacterial strain ATCC27853.

FIG. 9 illustrates that LpxCi-4 does not exhibit in vivo synergy with daptomycin in bacterial strain ATCC27853.

FIG. 10 illustrates that LpxCi-4 does not exhibit in vivo synergy with oxacillin in bacterial strain ATCC27853.

### DETAILED DESCRIPTION OF THE INVENTION

### Definitions

As used herein, the following definitions shall apply unless otherwise indicated.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “synergy” or “synergistic” as used herein means the combined effect of the compounds when used in combination is greater than the additive effects of the compounds when used individually. “Synergism” can be defined quantitatively as a fractional inhibitory concentration index (FICI) of ≦0.5, where FICI is defined as the sum of the fractional inhibitory concentrations (FICs) of the individual components in a combination of two compounds, and the FIC is defined as the ratio of the minimal inhibitory concentration (MIC) of the compound in the combination divided by the MIC of the compound alone:

$$F I C I = ( \frac{M I {C}_{drug A in combo}}{M I {C}_{drug A alone}} ) + ( \frac{M I {C}_{drug B in combo}}{M I {C}_{drug B alone}} )$$

Alternatively, “synergism,” more particularly “in vivo synergism,” can be defined quantitatively as an at least two-fold decrease in the static dose of the agents used in combination as compared to the LpxC inhibitor or the antibacterial agent alone. In certain cases one agent alone may never reach a static dose. In such cases, a combination is synergistic if bacterial growth can be halted (CFU load at 24 hours that is identical to that measured at 0 hours post infection) by combined administration with two compounds that alone cannot achieve stasis.

“LpxC” is an abbreviation that stands for UDP-3-O—(R-3-hydroxydecanoyl)-N-acetylglucosamine deacetylase.

The term “treating”, as used herein, refers to reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

“Co-administration” can be in the form of a single formulation (combining, for example, a LpxC inhibitor and an antibacterial agent with pharmaceutically acceptable excipients, optionally segregating the two active ingredients in different excipient mixtures designed to independently control their respective release rates and durations) or by independent administration of separate formulations containing the active agents. “Co-administration” further includes concurrent administration (administration of a LpxC inhibitor and an antibacterial agent at the same time) and time varied administration (administration of the LpxC inhibitor at a time different from that of the antibacterial agent), as long as both the LpxC inhibitor and antibacterial agent are present in the body in therapeutically effective concentrations during at least partially overlapping times.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkylene” refers to divalent saturated aliphatic hydrocarbyl groups preferably having from 1 to 6 and more preferably 1 to 3 carbon atoms that are either straight-chained or branched. This term is exemplified by groups such as methylene (—CH₂—), ethylene (—CH₂CH₂—), n-propylene (—CH₂CH₂CH₂—), iso-propylene (—CH₂CH(CH₃)—) or (—CH(CH₃)CH₂—), and the like.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Amino” refers to the group —NH₂.

“Aminocarbonyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like), provided that the point of attachment is through an atom of the aromatic aryl group. Preferred aryl groups include phenyl and naphthyl.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. Such groups are exemplified, for example, bi-vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

“Alkynyloxy” refers to the group —O-alkynyl, wherein alkynyl is as defined herein. Alkynyloxy includes, by way of example, ethynyloxy, propynyloxy, and the like.

“Carboxyl,” “carboxy” or “carboxylate” refers to —CO₂H or salts thereof.

“Cyano” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like.

“Cycloalkenyl” refers to non-aromatic cyclic alkyl groups of from 3 to 10 carbon atoms having single or multiple rings and having at least one double bond and preferably from 1 to 2 double bonds.

“Cycloalkynyl” refers to non-aromatic cycloalkyl groups of from 5 to 10 carbon atoms having single or multiple rings and having at least one triple bond.

“Halo” or “halogen” refers to fluoro, chloro, bromo, and iodo and is preferably fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen, and sulfur within the ring. Such heteroaryl groups can have a single ring (e.g., pyridinyl, imidazolyl or furyl) or multiple condensed rings (e.g., indolizinyl, quinolinyl, benzimidazolyl or benzothienyl), wherein the condensed rings may or may not be aromatic and/or contain a heteroatom, provided that the point of attachment is through an atom of the aromatic heteroaryl group. In one embodiment, the nitrogen and/or sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In one embodiment, the nitrogen and/or sulfur atom(s) of the heterocyclic group are optionally oxidized to provide for the N-oxide, —S(O)—, or —SO₂-moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

“Nitro” refers to the group —NO₂.

“Oxo” refers to the atom (═O).

“Sulfinyl” refers to the group —S(═O)-alkyl, —S(═O)-substituted alkyl, —S(═O)-alkenyl, —S(═O)-substituted alkenyl, —S(═O)-cycloalkyl, —S(═O)-substituted cycloalkyl, —S(═O)-cycloalkenyl, —S(═O)-substituted cycloalkenyl, —S(═O)-aryl, —S(═O)-substituted aryl, —S(═O)-heteroaryl, —S(═O)-substituted heteroaryl, —S(═O)-heterocyclic, and —S(═O)-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfinyl includes groups such as methyl-S(═O)—, phenyl-S(═O)—, and 4-methylphenyl-S(═O)—.

“Sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, and —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes groups such as methyl-SO₂—, phenyl-SO₂—, and 4-methylphenyl-SO₂—.

“Thiol” refers to the group —SH.

“Alkylthio” refers to the group —S-alkyl, wherein alkyl is as defined herein. In other embodiments, sulfur may be oxidized to —S(O)—. The sulfoxide may exist as one or more stereoisomers.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

The term “substituted,” when used to modify a specified group or radical, means that one or more hydrogen atoms of the specified group or radical are each, independently of one another, replaced with the same or different substituent groups as defined below.

Substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means e.g. that one of the counter ions for such divalent alkali earth ions can be an ionized form of a compound of the invention and the other a typical counter ion such as chloride, or two ionized compounds of the invention can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

Substituent groups for hydrogens on unsaturated carbon atoms in “substituted” alkenyl, alkynyl, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂⁻R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkenyl or alkynyl, the substituents are not —O⁻M⁺, —OR⁷⁰, —SR⁷⁰, or —S⁻M⁺.

Substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂⁻M⁺, —S(O)₂OR⁷⁰, —OS(O)₂R⁷⁰, —OS(O)₂⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁸⁰, R⁷⁰, R⁸⁰ and M⁺ are as previously defined.

In a preferred embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups are limited to substituted aryl-(substituted aryl)-substituted aryl.

“Stereoisomer” and “stereoisomers” refer to compounds that have same atomic connectivity but different atomic arrangement in space. Stereoisomers include cis-trans isomers, E and Z isomers, enantiomers, and diastereomers.

“Patient” refers to human and non-human animals, especially mammals.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like.

“Prodrug” refers to a derivative of an active compound (drug) that may require a transformation under the conditions of use, such as within the body, to release the active drug. Prodrugs are frequently, but not necessarily, pharmacologically inactive until converted into the active drug. Prodrugs are typically obtained by masking one or more functional groups in an active drug believed to be in part required for activity with a progroup (defined below) to form a promoiety which undergoes a transformation, such as cleavage, under the specified conditions of use to release the functional group, and hence the active drug. The cleavage of the promoiety may proceed spontaneously, such as by way of a hydrolysis reaction, or it can be catalyzed or induced by another agent, such as an enzyme, light, an acid or base, or a change of or exposure to a physical or environmental parameter, such as temperature. The agent can be endogenous to the conditions of use, such as an enzyme present in the cells to which the prodrug is administered or the acidic conditions of the stomach, or it can be supplied exogenously.

“Progroup” refers to a type of protecting group that, when used to mask a functional group within an active drug to form a promoiety, converts the drug into a prodrug. Progroups are typically attached to the functional group of the drug via bonds that are cleavable under specified conditions of use. Thus, a progroup is that portion of a promoiety that cleaves to release the functional group under the specified conditions of use. As a specific example, an amide promoiety of the formula —NH—C(O)CH₃ comprises the progroup —C(O)CH₃.

“Pharmaceutically effective amount” and “therapeutically effective amount” refer to an amount of the compound sufficient to treat bacterial infections, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water.

It is understood that the above definitions are not intended to include impermissible substitution patterns (e.g., methyl substituted with 5 fluoro groups). Such impermissible substitution patterns are easily recognized by a person having ordinary skill in the art.

### Antibiotic Combinations

The present invention provides novel combinations of compounds including at least one LpxC inhibitor, as well as methods for treating subjects infected with gram-negative bacteria. The novel combinations provided herein can be formulated into pharmaceutical formulations and medicaments that are useful in the methods of the invention. The invention also provides for the use of the novel combinations in preparing medicaments and pharmaceutical formulations, for use of the combinations in treating bacterial infections in a patient.

One classic method for assessing synergy, referred to as the checkerboard assay, is used to predict the efficacy of antibacterial agents, and is described by Scribner et. al., (Antimicrobial Agents and Chemotherapy 21(6):939-943 (1982)) and in Goodman & Gilman (The Pharmacological Basis of Therapeutics, Sixth Edition, pp. 1097-1098 (1980)). The checkerboard assay involves serial two-fold dilutions of the antibiotics individually and in combination in broth, which is then inoculated with the microorganism to be tested. After incubation, the minimum inhibitory concentration (MIC) of each drug used individually and in combination is determined (N.B., the MIC is the lowest concentration of the drug that inhibits growth in the medium). Synergism is indicated by a decrease in the MIC of each drug when used in combination. Antagonism is indicated by an increase in the MIC of either or both drugs when used in combination. Alternate methods of assessing synergy are reviewed in Greco, et al., Pharmacological Reviews 47(2):331-285 (1995), incorporated herein by reference in its entirety.

Surprisingly, the present invention demonstrates that a positive result in a checkerboard assay, i.e., indicating synergy below the MIC, does not necessarily result in synergistic behavior in vivo. For example, U.S. Patent Application Publication No. 2004-229955A1 reports strong synergy between erythromycin and an LpxC inhibitor, N-[(1S)-1-(aminomethyl)-2-(hydroxyamino)-2-oxoethyl]-4-(4-{4-[({[(3methylphenyl)methyl]amino}acetyl)amino]phenyl}buta-1,3-diynyl)benzamide against E. coli strain ATCC 25922. However, as demonstrated in Example 3, below, the combination of erythromycin and an LpxC inhibitor shows no synergy in vivo.

LpxC, an essential gene in gram-negative bacteria, encodes the enzyme uridyldiphospho-3-O—(R-hydroxydecanoyl)-N-acetylglucosamine deacetylase. This enzyme catalyzes an early committed step in the bio-synthesis of lipid A, the lipid moiety of lipopolysaccharide, which is an essential component of all gram-negative bacteria. Above the MIC, an LpxC inhibitor is expected to disrupt the outer membrane, thus permitting other antibacterial compounds to penetrate the outer membrane. Once these agents have penetrated the outer membrane, they may affect periplasmic targets as is the case with vancomycin, or they may then diffuse across the inner membrane to interact with an intracellular target such as the ribosome (erythromycin) or RNA polymerase (rifampin). In the absence of an LpxC inhibitor, the ability of agents such as vancomycin to access their target is greatly diminished by the outer membrane. Thus, the biochemical mechanism that we believe underlies the observed synergy is the enhanced permeability of the outer membrane to agents such as vancomycin when combined with LpxC inhibitors. Indeed, as demonstrated in Example 1, below, exposure to an LpxC inhibitor mimics the membrane disrupting effects of an imp mutation in gram-negative bacteria, allowing entrance to compounds that would otherwise be excluded by the outer membrane.

In one embodiment, the antibacterial agent used in combination with an LpxC inhibitor is selected from the group consisting of vancomycin, linezolid, azithromycin, imipenem, teicoplanin, daptomycin, clindamycin, rifampin, cefotaxime, gentamicin, novobiocin, and telavancin. In a more specific embodiment, the antibacterial agent is vancomycin, teicoplanin, rifampin, azithromycin, telavancin or novobiocin. In a yet more specific embodiment, the antibacterial agent is vancomycin or rifampin. In some embodiments of the invention, the antibacterial agent and/or the LpxC inhibitor is administered at a sub-therapeutic dose, wherein a subtherapeutic dose is a dose that would be insufficient to treat bacterial infections, if administered alone.

The compositions and methods of the present invention can utilize a compound capable of inhibiting LpxC. In one embodiment, the LpxC inhibitor can be selected from compounds having formula (I):

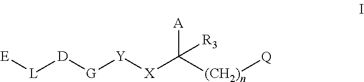

or a stereoisomer, pharmaceutically acceptable salt, ester, or prodrug thereof, wherein

E is absent or selected from the group consisting of
(1) H,
(2) substituted or unsubstituted C₁-C₆-alkyl,
(3) substituted or unsubstituted C₂-C₆-alkenyl,
(4) substituted or unsubstituted C₂-C₆-alkynyl,
(5) substituted or unsubstituted aryl,
(6) substituted or unsubstituted heterocyclyl, and
(7) substituted or unsubstituted heteroaryl;

L is absent or selected from the group consisting of
(1) substituted or unsubstituted C₁-C₆-alkyl,
(2) —(NH)₀₋₁—(CH₂)ⱼ—NR^{3L}—(CH₂)ₖ—,
(3) —(NH)₀₋₁—C(R^{1L},R^{2L})—NR^{3L}—C(R^{1L},R^{2L})—,
(4) —C(R^{1L},R^{2L})—O—C(R^{1L},R^{2L})—,
(5) —(CH₂)ⱼ—NR^{3L}—C(R^{1L},R^{2L})—CONH—(CH₂)ₖ—,
(6) —CO—C(R^{1L},R^{2L})—NHCO—,
(7) —CONH—, and
(8) —NHCO—,
   wherein R^{1L},R^{2L}, and R^{3L} are independently selected from the group consisting of (a) H, (b) substituted or unsubstituted C₁-C₆-alkyl, (c) C₁-C₆-alkyl substituted with aryl, (d) C₁-C₆-alkyl substituted with heterocyclyl, and (e) C₁-C₆-alkyl substituted with heteroaryl, or R^{1L} and R^{3L}, together with the atoms to which they are attached can form a substituted or unsubstituted heterocyclic ring, having from 3 to 8 ring atoms, wherein 1-2 ring atoms of the heterocyclic ring system are selected from N, O and S;

j is an integer of 0-4;

k is an integer of 0-4;

D is absent or selected from the group consisting of
(1) substituted or unsubstituted C₃-C₈-cycloalkyl,
(2) substituted or unsubstituted aryl,
(3) substituted or unsubstituted heterocyclyl, and
(4) substituted or unsubstituted heteroaryl;

G is absent or selected from the group consisting of
(1) —(CH₂)ᵢ—O—(CH₂)ᵢ—,
(2) —(CH₂)ᵢ—S—(CH₂)ᵢ—,
(3) —(CH₂)ᵢ—NR^{g}—(CH₂)ᵢ—,
(4) —C(═O)—,
(5) —NHC(═O)—,
(6) —C(═O)NH—,
(7) —(CH₂)ᵢNHCH₂C(═O)NH—,
(8) —C≡C—,
(9) —C≡C—C≡C—, and
(10) —C═C—;
   wherein R^{g} is H or substituted or unsubstituted C₁-C₆-alkyl;
i is an integer of 0-4;

Y is selected from the group consisting of
(1) substituted or unsubstituted C₃-C₈-cycloalkyl,
(2) substituted or unsubstituted aryl,
(3) substituted or unsubstituted heterocyclyl, and
(4) substituted or unsubstituted heteroaryl;

X is selected from the group consisting of
(1) —(C═O)—,
(2) —C₁-C₆-alkyl-(C═O)C—,
(3) —C₂-C₆-alkenyl-(C═O)—,
(4) —C₂-C₆-alkynyl-(C═O)—, and
(5) —CH₂—;
   or when B is absent, X and A, together with the atoms to which they are attached can form a heterocyclic ring, having from 5 to 8 ring atoms, wherein 1-2 ring atoms of the heterocyclic ring system are selected from N, O and S;

B is absent or

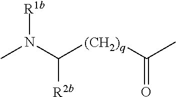

wherein R^{1b} and R^{2b}, are independently selected from the group consisting of (a) H, (b) substituted or unsubstituted C₁-C₆-alkyl, (c) substituted or unsubstituted C₂-C₆-alkenyl, (d) substituted or unsubstituted C₂-C₆-alkynyl, (e) substituted or unsubstituted aryl, (f) substituted or unsubstituted heterocyclyl, (g) substituted or unsubstituted heteroaryl, (h) C₁-C₆-alkyl substituted with aryl, (i) C₁-C₆-alkyl substituted with heterocyclyl, and (j) C₁-C₆-alkyl substituted with heteroaryl,
or R^{1b} and R^{2b}, together with the atoms to which they are attached can form a substituted or unsubstituted heterocyclic ring, having from 5 to 8 ring atoms, wherein 1-2 ring atoms of the heterocyclic ring system are selected from N, O and S;

q is an integer of 0-4;

R₃ is H or substituted or unsubstituted C₁-C₆-alkyl,

or R₃ and A, together with the atoms to which they are attached can form a substituted or unsubstituted 3-10 membered cycloalkyl or a heterocyclic ring system, wherein the heterocyclic ring system may have from 3 to 10 ring atoms, with 1 to 2 rings being in the ring system and contain from 1-4 heteroatoms selected from N, O and S;

R⁴ is H or substituted or unsubstituted C₁-C₆-alkyl,

or R₄ and A, together with the atoms to which they are attached can form a substituted or unsubstituted heterocyclic ring, having from 3 to 8 ring atoms, wherein 1-2 ring atoms of the heterocyclic ring system are selected from N, O and S; n is an integer of 0-6;

A is selected from the group consisting of
(1) H,
(2) —(CH₂)ᵣC(R^{1a},R^{2a})(CH₂) OR^{3a},
(3) —(CH₂)ᵣC(R^{1a},R^{2a})N(R^{4a},R^{5a}),
(4) —(CH₂)ᵣC(R^{1a},R^{2a})N(R^{4a})COR^{3a},
(5) —(CH₂)ᵣC(R^{1a},R^{2a})NHCON(R^{4a},R^{5a}),
(6) —(CH₂)ᵣC(R^{1a},R^{2a})NHC(═NH)N(R^{4a},R^{5a}),
(7) —CH(R^{1a},R^{2a}),
(8) —C≡CH,
(9) —(CH₂)ᵣC(R^{1a},R^{2a})CN,
(10) —(CH₂)ᵣC(R^{1a},R^{2a})CO₂R^{3a}, and
(11) —(CH₂)ᵣC(R^{1a},R^{2a})CON(R^{4a},R^{5a}),
   wherein R^{1a}, R^{2a}, R^{3a}, R^{4a}, and R^{5a} are independently selected from the group consisting of (a) H, (b) substituted or unsubstituted C₁-C₆-alkyl, (c) substituted or unsubstituted aryl, (d) substituted or unsubstituted heterocyclyl, (e) substituted or unsubstituted heteroaryl, (f) C₁-C₆-alkyl substituted with aryl, (g) C₁-C₆-alkyl substituted with heterocyclyl, and (h) C₁-C₆-alkyl substituted with heteroaryl, or R^{4a} and R^{5a} together with the N atom to which they are attached can form a substituted or unsubstituted heterocyclic ring, having from 5 to 8 ring atoms, wherein 1-2 ring atoms of the heterocyclic ring system are selected from N, O and S;
r is an integer of 0-4;
s is an integer of 0-4;
   Q is absent or selected from the group consisting of
(1) —C(═O)N(R₁,R₂),
(2) —NHC(═O)N(R₁,R₂),
(3) —N(OH)C(═O)N(R₁,R₂),
(4) —CH(OH)C(═O)N(R₁,R₂),
(5) —CH[N(R^{2q}, R^{3q})]C(═O)N(R₁,R₂),
(6) —CHR^{1q}C(═O)N(R₁,R₂),
(7) —CO₂H,
(8) —C(═O)NHSO₂R^{4q},
(9) —SO₂NH₂,
(10) —N(OH)C(═O)R^{1q},
(11) —N(OH)SO₂R^{4q},
(12) —NHSO₂R^{4q},
(13) —SH,
(14) —CH(SH)(CH₂)₀₋₁C(═O)N(R₁,R₂),
(15) —CH(SH)(CH₂)₀₋₁CO₂H,
(16) —CH(OH)(CH₂)₀₋₁CO₂H,
(17) —CH(SH)CH₂CO₂R^{1q},
(18) —CH(OH)(CH₂)SO₂NH₂,
(19) —CH(CH₂SH)NHCOR^{1q},
(20) —H(CH₂SH)NHSO₂R^{4q},
(21) —CH(CH₂SR^{5q})CO₂H,
(22) —CH(CH₂SH)NHSO₂NH₂,
(23) —CH(CH₂OH)CO₂H,
(24) —H(CH₂OH)NHSO₂NH₂,
(25) —C(═O)CH₂CO₂H,
(26) —C(═O)(CH₂)₀₋₁CONH₂,
(27) —OSO₂NHR^{5q},
(28) —SO₂NHNH₂,
(29) —P(═O)(OH)₂,

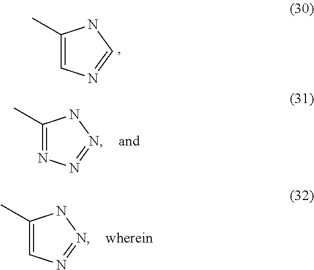

R₁ is selected from the group consisting of (1) H, (2) —OH, (3) —OC₁₋₆-alkyl, (4) —N(R^{2q},R^{3q}), and (5) substituted or unsubstituted C₁-C₆-alkyl;
R₂ is selected from the group consisting of
(1) H,
(2) substituted or unsubstituted C₁-C₆-alkyl,
(3) substituted or unsubstituted C₂-C₆-alkenyl,
(4) substituted or unsubstituted C₂-C₆-alkenyl,
(5) substituted or unsubstituted aryl,
(6) substituted or unsubstituted heterocyclyl,
(7) substituted or unsubstituted heteroaryl,
(8) C₁-C₆-alkyl substituted with aryl,
(9) C₁-C₆-alkyl substituted with heterocyclyl, and
(10) C₁-C₆-alkyl substituted with heteroaryl,
   or R¹ and R², together with the N atom to which they are attached can form a substituted or unsubstituted heterocyclic ring, having from 3 to 10 ring atoms, wherein 1-4 ring atoms of the heterocyclic ring system are selected from N, O and S, or R² and R⁴, together with the N atoms to which they are attached can form a substituted or unsubstituted heterocyclic ring, having from 3 to 10 ring atoms, wherein 1-4 ring atoms of the heterocyclic ring system are selected from N, O and S;
   R^{1q}, R^{2q}, R^{3q}, R^{4q}, and R^{5q} are selected from H or C₁-C₆ alkyl,
   wherein B is absent, or E, L, G, and B are absent, or E, L, and G are absent, or E, L, and B are absent, or E, L, D, G, and B are absent.

In another embodiment, the LpxC inhibitor can be selected from compounds having formula (I):

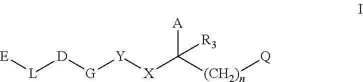

including stereoisomers, pharmaceutically acceptable salts, esters, and prodrugs thereof, wherein:
E is selected from the group consisting of:
   (1) H,
   (2) substituted or unsubstituted C₁-C₆-alkyl,
   (3) substituted or unsubstituted C₂-C₆-alkenyl,
   (4) substituted or unsubstituted C₂-C₆-alkynyl,
   (5) substituted or unsubstituted C₃-C₁₀-cycloalkyl,
   (6) substituted or unsubstituted aryl,
   (7) substituted or unsubstituted heterocyclyl, and
   (8) substituted or unsubstituted heteroaryl;
L is absent or selected from the group consisting of:
   (1) substituted or unsubstituted C₁-C₆-alkyl,
   (2) —(NR^{3L})₀₋₁—(CH₂)₀₋₄—NR^{3L}—(CH₂)₀₋₄—,
   (3) —(NR^{3L})₀₋₁—C(R^{1L},R^{2L})—NR^{3L}-—C(R^{1L},R^{2L})—,
   (4) —C(R^{1L},R^{2L})—O—C(R^{1L}R^{2L})—,
   (5) —(CH₂)₀₋₄—NR^{3L}—C(R^{1L},R^{2L})—CONH—(CH₂)₀₋₄—,
   (6) —CO—C(R^{1L},R^{2L})—NHCO—,
   (7) —CONR^{3L}—,
   (8) —NR^{3L}—,
   (9) —NR^{3L}—,
   (10) —SO₂NR^{3L}—,
   (11) —NR^{3L}—C(═O)—NR^{3L}—,
   (12) substituted or unsubstituted C₃-C₁₀-cycloalkyl,
   (13) substituted or unsubstituted aryl,
   (14) substituted or unsubstituted heterocyclyl, and
   (15) substituted or unsubstituted heteroaryl,
   wherein:
      each R^{1L},R^{2L}, and R^{3L} is independently selected from the group consisting of:
         (a) H,
         (b) substituted or unsubstituted C₁-C₆-alkyl,
         (c) C₁-C₆-alkyl substituted with aryl,
         (d) C₁-C₆-alkyl substituted with heterocyclyl, and
         (e) C₁-C₆-alkyl substituted with heteroaryl,
   or R^{1L} and R^{3L}, together with the atoms to which they are attached can form a substituted or unsubstituted heterocyclic ring, having from 3 to 8 ring atoms, wherein 1-2 ring atoms of the heterocyclic ring are selected from N, O and S;
D is absent or selected from the group consisting of:
   (1) substituted or unsubstituted C₃-C₁₀-cycloalkyl,
   (2) substituted or unsubstituted aryl,
   (3) substituted or unsubstituted heterocyclyl, and
   (4) substituted or unsubstituted heteroaryl;
G is selected from the group consisting of:
   (1) —NR^{1G}C(═O)—,
   (2) —C(═O)NR^{1G}—,
   (3) —(CH₂)₀₋₄NHCH₂C(═O)NR^{1G}—,
   (4) —CR^{2G}═CR^{2G}—,
   (5) —S(═O)—,
   (6) —SO₂—,
   (7) —C(R^{3G})₂—S(═O)—,
   (8) —S(═O)—C(R^{3G})₂—,
   (9) —C(R^{3G})₂—SO₂—,
   (10) —SO₂—C(R^{3G})₂—
   (11) —CR^{3G}═CR^{3G}—CR^{3G}═CR^{3G}—,
   (12) —C(R^{3G})₂—,
   (13) —CR^{3G}═CR^{3G}—C≡C—,
   (14) —C≡C—CR^{3G}═CR^{3G}—,
   (15) —C(═O)—C≡C—,
   (16) —C≡C—C(═O)—,
   (17) substituted or unsubstituted C₃-C₁₀-cycloalkyl,
   (18) substituted or unsubstituted aryl,
   (19) substituted or unsubstituted heterocyclyl, and
   (20) substituted or unsubstituted heteroaryl,
   wherein:
      R^{1G} is substituted or unsubstituted C₁-C₆-alkyl;
      each R^{2G} is independently selected from the group consisting of H, a halogen atom, and substituted or unsubstituted C₁-C₆-alkyl, and at least one R^{2G} is not H; and
      R^{3G} is selected from the group consisting of H, a halogen atom, and substituted or unsubstituted C₁-C₆-alkyl;
Y is absent or selected from the group consisting of:
   (1) substituted or unsubstituted C₃-C₁₀-cycloalkyl,
   (2) substituted or unsubstituted aryl,
   (3) substituted or unsubstituted heterocyclyl, and
   (4) substituted or unsubstituted heteroaryl;
X is selected from the group consisting of:
   (1) —(C═O)NR₄—,
   (2) —C₁-C₆-alkyl-(C═O)NR₄—,
   (3) —C₂-C₆-alkenyl-(C═O)NR₄—,
   (4) —C₂-C₆-alkynyl-(C═O)NR₄—,
   (5) —CH₂NR₄—,
   (6) —SO₂NR₄—,
   (7) —S(═O)NR₄—,
   (8) —NR₄C(═O)—, and
   (9) —NR₄—,
   or X and A, together with the atoms to which they are attached can form a heterocyclic ring, having from 5 to 8 ring atoms, wherein 1-2 ring atoms of the heterocyclic ring are selected from N, O and S,
   or when Y is a bicyclic substituted or unsubstituted heterocyclyl or heteroaryl, then X is absent;
R₃ is H or substituted or unsubstituted C₁-C₆-alkyl, or R₃ and A, together with the atom to which they are attached can form a substituted or unsubstituted 3-10 membered cycloalkyl or a heterocyclic ring, having from 3 to 10 ring atoms, wherein 1-4 ring atoms of the heterocyclic ring are selected from N, O and S;
R₄ is (1) H or substituted or unsubstituted C₁-C₆-alkyl, or (2) R₄ and A, together with the atoms to which they are attached can form a substituted or unsubstituted heterocyclic ring, having from 3 to 8 ring atoms, wherein 1-2 ring atoms of the heterocyclic ring are selected from N, O and S, or (3) R₄ and Y, together with the atoms to which they are attached, form a bicyclic substituted or unsubstituted heterocyclyl or heteroaryl;
n is an integer from 0-6;
A is selected from the group consisting of:
   (1) H,
   (2) —(CH₂)₀₋₄C(R^{1a},R^{2a})(CH₂)₀₋₄OR^{3a},
   (3) —(CH₂)₀₋₄C(R^{1a},R^{2a})N(R^{4a},R^{5a}),
   (4) —(CH₂)₀₋₄C(R^{1a},R^{2a})N(R^{4a})COR^{3a},
   (5) —(CH₂)₀₋₄C(R^{1a},R^{2a})NHCON(R^{4a},R^{5a}),
   (6) —(CH₂)₀₋₄C(R^{1a},R^{2a})NHC(═NH)N(R^{4a},R^{5a}),
   (7) —CH(R^{1a},R^{2a}),
   (8) —C≡CH,
   (9) —(CH₂)₀₋₄C(R^{1a},R^{2a})CN,
   (10) —(CH₂)₀₋₄C(R^{1a},R^{2a})CO₂R^{3a},
   (11) —(CH₂)₀₋₄C(R^{1a},R^{2a})CON(R^{4a},R^{5a}),
   (12) substituted or unsubstituted C₃-C₁₀-cycloalkyl,
   (13) substituted or unsubstituted aryl,
   (14) substituted or unsubstituted heterocyclyl, and
   (15) substituted or unsubstituted heteroaryl,
   wherein:
      each R^{1a}, R^{2a}, R^{3a}, R^{4a}, and R^{5a} is independently selected from the group consisting of:
         (a) H,
         (b) a halogen atom,
         (c) substituted or unsubstituted C₁-C₆-alkyl,
         (d) substituted or unsubstituted aryl,
         (e) substituted or unsubstituted heterocyclyl, and
         (f) substituted or unsubstituted heteroaryl,
   or R^{4a} and R^{5a} together with the N atom to which they are attached can form a substituted or unsubstituted heterocyclic ring, having from 5 to 8 ring atoms, wherein 1-2 ring atoms of the heterocyclic ring are selected from N, O and S;
Q is absent or selected from the group consisting of:
   (1) —C(═O)N(R₁,R₂),
   (2) —NHC(═O)N(R₁,R₂),
   (3) —N(OH)C(═O)N(R₁,R₂),
   (4) —CH(OH)C(═O)N(R₁,R₂),
   (5) —CH[N(R^{2q},R^{3q})]C(═O)N(R₁,R₂),
   (6) —CHR^{1q}C(═O)N(R₁,R₂),
   (7) —CO₂H,
   (8) —C(═O)NHSO₂R^{4q},
   (9) —SO₂NH₂,
   (10) —N(OH)C(═O)R^{1q},
   (11) —N(OH)SO₂R^{4q},
   (12) —NHSO₂R^{4q},
   (13) —SH,
   (14) —CH(SH)(CH₂)₀₋₁C(═O)N(R₁,R₂),
   (15) —CH(SH)(CH₂)₀₋₁CO₂H,
   (16) —CH(OH)(CH₂)₀₋₁CO₂H,
   (17) —CH(SH)CH₂CO₂R^{1q},
   (18) —CH(OH)(CH₂)SO₂NH₂,
   (19) —CH(CH₂SH)NHCOR^{1q},
   (20) —CH(CH₂SH)NHSO₂R^{4q},
   (21) —CH(CH₂SR^{5q})CO₂H,
   (22) —CH(CH₂SH)NHSO₂NH₂,
   (23) —CH(CH₂OH)CO₂H,
   (24) —CH(CH₂OH)NHSO₂NH₂,
   (25) —C(═O)CH₂CO₂H,
   (26) —C(═O)(CH₂)₀₋₁CONH₂,
   (27) —OSO₂NHR^{5q},
   (28) —SO₂NHNH₂,
   (29) —P(═O)(OH)₂,

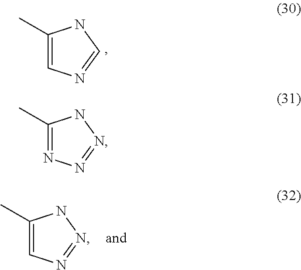

(33) —N(OH)C(═O)CR₁R₂,
wherein:
   R₁ is selected from the group consisting of:
      (1) —H,
      (2) —OH,
      (3) —OC₁-C₆-alkyl,
      (4) —N(R^{2q},R^{3q}), and
      (5) substituted or unsubstituted C₁-C₆-alkyl;
   R₂ is selected from the group consisting of:
      (1) H,
      (2) substituted or unsubstituted C₁-C₆-alkyl,
      (3) substituted or unsubstituted C₂-C₆-alkenyl,
      (4) substituted or unsubstituted C₂-C₆-alkenyl,
      (5) substituted or unsubstituted aryl,
      (6) substituted or unsubstituted heterocyclyl, and
      (7) substituted or unsubstituted heteroaryl,
   or R₁ and R₂, together with the N atom to which they are attached can form a substituted or unsubstituted heterocyclic ring, having from 3 to 10 ring atoms, wherein 1-4 ring atoms of the heterocyclic ring are selected from N, O and S; and
   each R^{1q}, R^{2q}, R^{3q}, R^{4q}, and R^{5q} is independently selected from the group consisting of H and C₁-C₆ alkyl.

In another embodiment, the LpxC inhibitor can be selected from compounds having formula (I):

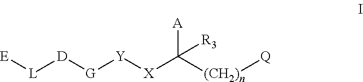

including stereoisomers, pharmaceutically acceptable salts, esters, and prodrugs thereof, wherein:
E is selected from the group consisting of:
   (1) H,
   (2) substituted or unsubstituted C₁-C₆-alkyl,
   (3) substituted or unsubstituted C₂-C₆-alkenyl,
   (4) substituted or unsubstituted C₂-C₆-alkynyl,
   (5) substituted or unsubstituted C₃-C₁₀-cycloalkyl,
   (6) substituted or unsubstituted aryl,
   (7) substituted or unsubstituted heterocyclyl, and
   (8) substituted or unsubstituted heteroaryl;
L is absent or selected from the group consisting of:
   (1) substituted or unsubstituted
   (2) —(NR^{3L})₀₋₁—(CH₂)₀₋₄—NR^{3L}—(CH₂)₀₋₄—,
   (3) —(NR^{3L})₀₋₁—C(R^{1L},R^{2L})—NR^{3L}—C(R^{1L},R^{2L})—,
   (4) —C(R^{1L},R^{2L})—O—C(R^{1L},R^{2L})—,
   (5) —(CH₂)₀₋₄—NR^{3L}—C(R^{1L},R^{2L})—CONH—(CH₂)₀₋₄—,
   (6) —CO—C(R^{1L},R^{2L})—NHCO—,
   (7) —CONR^{3L}—,
   (8) —NR^{3L}CO—,
   (9) —NR^{3L}—,
   (10) —SO₂NR^{3L}—,
   (11) —NR^{3L}—C(═O)—NR^{3L}—,
   (12) substituted or unsubstituted C₃-C₁₀-cycloalkyl,
   (13) substituted or unsubstituted aryl,
   (14) substituted or unsubstituted heterocyclyl, and
   (15) substituted or unsubstituted heteroaryl,
   wherein:
      each R^{1L},R^{2L}, and R^{3L} is independently selected from the group consisting of:
         (a) H,
         (b) substituted or unsubstituted C₁-C₆-alkyl,
         (c) C₁-C₆-alkyl substituted with aryl,
         (d) C₁-C₆-alkyl substituted with heterocyclyl, and
         (e) C₁-C₆-alkyl substituted with heteroaryl,
   or R^{1L} and R^{3L}, together with the atoms to which they are attached can form a substituted or unsubstituted heterocyclic ring, having from 3 to 8 ring atoms, wherein 1-2 ring atoms of the heterocyclic ring are selected from N, O and S;
D is absent or selected from the group consisting of:
   (1) substituted or unsubstituted C₃-C₁₀-cycloalkyl,
   (2) substituted or unsubstituted aryl,
   (3) substituted or unsubstituted heterocyclyl, and
   (4) substituted or unsubstituted heteroaryl;
G is selected from the group consisting of:
   (1) —(CH₂)₀₋₄—O—(CH₂)₀₋₄—,
   (2) —(CH₂)₀₋₄—S—(CH₂)₀₋₄—,
   (3) —(CH₂)₀₋₄—NR^{1G}—(CH₂)₀₋₄—,
   (4) —C(═O)—,
   (5) —NR^{1G}C(═O)—,
   (6) —C(═O)NR^{1G}—,
   (7) —(CH₂)₀₋₄NHCH₂C(═O)NR^{1G}—,
   (8) —C≡C—,
   (9) —C≡C—C≡C—,
   (10) —CR^{2G}═CR^{2G}—,
   (11) —S(═O)—,
   (12) —SO₂—,
   (13) —C(R^{3G})₂—S(═O)—,
   (14) —S(═O)—C(R^{3G})₂—,
   (15) —C(R^{3G})₂—SO₂—,
   (16) —SO₂—C(R^{3G})₂—
   (17) —CR^{3G}═CR^{3G}—CR^{3G}═CR^{3G}—,
   (18) —C(R^{3G})₂—,
   (19) —CR^{3G}═CR^{3G}—C≡C—,
   (20) —C≡C—CR^{3G}═CR^{3G}—,
   (21) —C(═O)—C≡C—,
   (22) —C≡C—C(═O)—,
   (23) substituted or unsubstituted C₃-C₁₀-cycloalkyl,
   (24) substituted or unsubstituted aryl,
   (25) substituted or unsubstituted heterocyclyl, and
   (26) substituted or unsubstituted heteroaryl,
   wherein:
      R^{1G} is substituted or unsubstituted C₁-C₆-alkyl;
      each R^{2G} and R^{3G} is independently selected from the group consisting of H, a halogen atom, and substituted or unsubstituted C₁-C₆-alkyl;
Y is absent or selected from the group consisting of:
   (1) substituted or unsubstituted C₃-C₁₀-cycloalkyl,
   (2) substituted or unsubstituted aryl,
   (3) substituted or unsubstituted heterocyclyl, and
   (4) substituted or unsubstituted heteroaryl;
X is selected from the group consisting of:
   (1) —(C═O)NR₄—,
   (2) —C₁-C₆-alkyl-(C═O)NR₄—,
   (3) —C₂-C₆-alkenyl-(C═O)NR₄—,
   (4) —C₂-C₆-alkynyl-(C═O)NR₄—,
   (5) —CH₂NR₄—,
   (6) —SO₂NR₄—,
   (7) —S(═O)NR₄—,
   (8) —NR₄C(═O)—, and
   (9) —NR₄—,
   or X and A, together with the atoms to which they are attached can form a heterocyclic ring, having from 5 to 8 ring atoms, wherein 1-2 ring atoms of the heterocyclic ring are selected from N, O and S,
   or when Y is a bicyclic substituted or unsubstituted heterocyclyl or heteroaryl, then X is absent;
R₃ is H or substituted or unsubstituted C₁-C₆-alkyl, or R₃ and A, together with the atom to which they are attached can form a substituted or unsubstituted 3-10 membered cycloalkyl or a heterocyclic ring, having from 3 to 10 ring atoms, wherein 1-4 ring atoms of the heterocyclic ring are selected from N, O and S;
R₄ is (1) H or substituted or unsubstituted C₁-C₆-alkyl, or (2) R₄ and A, together with the atoms to which they are attached can form a substituted or unsubstituted heterocyclic ring, having from 3 to 8 ring atoms, wherein 1-2 ring atoms of the heterocyclic ring are selected from N, O and S, or (3) R₄ and Y, together with the atoms to which they are attached, form a bicyclic substituted or unsubstituted heterocyclyl or heteroaryl;
n is an integer from 0-6;
A is selected from the group consisting of:
   (1) —C(R^{1a},R^{2a})OR^{3a},
   (2) —C(R^{1a},R^{2a})N(R^{4a},R^{5a}),
   (3) substituted or unsubstituted C₃-C₁₀-cycloalkyl,
   (4) substituted or unsubstituted aryl,
   (5) substituted or unsubstituted heterocyclyl, and
   (6) substituted or unsubstituted heteroaryl,
   wherein:
      each R^{1a} and R^{2a} is independently selected from the group consisting of substituted or unsubstituted C₁-C₆-alkyl;
      each R^{3a}, R^{4a}, and R^{5a} is independently selected from the group consisting of:
         (a) H,
         (b) a halogen atom,
         (c) substituted or unsubstituted C₁-C₆-alkyl,
         (d) substituted or unsubstituted aryl,
         (e) substituted or unsubstituted heterocyclyl, and
         (f) substituted or unsubstituted heteroaryl,
   or R^{4a} and R^{5a} together with the N atom to which they are attached can form a substituted or unsubstituted heterocyclic ring, having from 5 to 8 ring atoms, wherein 1-2 ring atoms of the heterocyclic ring are selected from N, O and S; and
      when A is —C(R^{1a},R^{2a})OR^{3a}, the compound is not 2-{[(4′-ethyl-1,1′-biphenyl-4-yl)carbonyl]amino}-3-hydroxy-3-methylbutanoic acid, 4′-ethyl-N-{2-hydroxy-1-[(hydroxyamino)carbonyl]-2-methylpropyl}-1,1′-biphenyl-4-carboxamide or N-{2-hydroxy-1-[(hydroxyamino)carbonyl]-2-methylpropyl}-4-(phenylethynyl)benzamide;
Q is absent or selected from the group consisting of:
   (1) —C(═O)N(R₁,R₂),
   (2) —NHC(═O)N(R₁,R₂),
   (3) —N(OH)C(═O)N(R₁,R₂),
   (4) —CH(OH)C(═O)N(R₁,R₂),
   (5) —CH[N(R^{2q},R^{3q})]C(═O)N(R₁,R₂),
   (6) —CHR^{1q}C(═O)N(R₁,R₂),
   (7) —CO₂H,
   (8) —C(═O)NHSO₂R^{4q},
   (9) —SO₂NH₂,
   (10) —N(OH)C(═O)R^{1q},
   (11) —N(OH)SO₂R^{4q},
   (12) —NHSO₂R^{4q},
   (13) —SH,
   (14) —CH(SH)(CH₂)₀₋₁C(═O)N(R₁,R₂),
   (15) —CH(SH)(CH₂)₀₋₁CO₂H,
   (16) —CH(OH)(CH₂)₀₋₁CO₂H,
   (17) —CH(SH)CH₂CO₂R^{1q},
   (18) —CH(OH)(CH₂)SO₂NH₂,
   (19) —CH(CH₂SH)NHCOR^{1q},
   (20) —CH(CH₂SH)NHSO₂R^{4q},
   (21) —CH(CH₂SR^{5q})CO₂H,
   (22) —CH(CH₂SH)NHSO₂NH₂,
   (23) —CH(CH₂OH)CO₂H,
   (24) —CH(CH₂OH)NHSO₂NH₂,
   (25) —C(═O)CH₂CO₂H,
   (26) —C(═O)(CH₂)₀₋₁CONH₂,
   (27) —OSO₂NHR^{5q},
   (28) —SO₂NHNH₂,
   (29) —P(═O)(OH)₂,

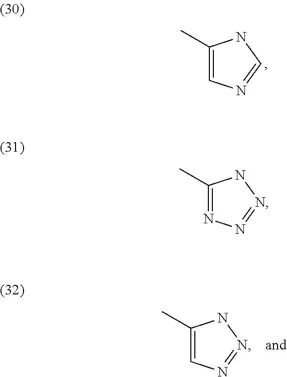

(33) —N(OH)C(═O)CR₁R₂,
wherein:
   R₁ is selected from the group consisting of:
      (1) —H,
      (2) —OH,
      (3) —OC₁-C₆-alkyl,
      (4) —N(R^{2q},R^{3q}), and
      (5) substituted or unsubstituted C₁-C₆-alkyl;
   R₂ is selected from the group consisting of:
      (1) H,
      (2) substituted or unsubstituted C₁-C₆-alkyl,
      (3) substituted or unsubstituted C₂-C₆-alkenyl,
      (4) substituted or unsubstituted C₂-C₆-alkenyl,
      (5) substituted or unsubstituted aryl,
      (6) substituted or unsubstituted heterocyclyl, and
      (7) substituted or unsubstituted heteroaryl,
   or R₁ and R₂, together with the N atom to which they are attached can form a substituted or unsubstituted heterocyclic ring, having from 3 to 10 ring atoms, wherein 1-4 ring atoms of the heterocyclic ring are selected from N, O and S; and
   each R^{1q}, R^{2q}, R^{3q}, R^{4q}, and R^{5q} is independently selected from the group consisting of H and C₁-C₆ alkyl.

In another embodiment, the LpxC inhibitor can be selected from compounds having formula II-A, II-B or II-C:

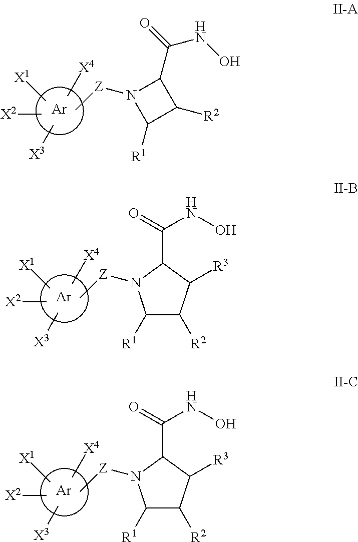

wherein:
X¹, X², X³, and X⁴ are independently selected from the group consisting of hydrogen, alkyl, haloalkyl, alkylthio, alkylsulfinyl, alkylsulfonyl, hydroxyalkyl, alkoxy, alkoxyalkyl, haloalkoxy, alkenyl, alkenoxy, alkenoxyalkyl, alkynyl, alkynyloxy, nitro, halo, hydroxy, cycloalkyl, cycloalkylalkyl, arylalkoxy, arylalkoxyalkyl, haloalkylthio, haloalkylsulfinyl, haloalkylsulfonyl, haloarylalkyl, haloarylalkynyl, alkylsilylalkynyl, aryl, alkynyloxy, anaminocarbonylalkyl, carboxylate, carboxyl, carboxamide, heterocycle, and substituted heterocycle;
R¹ and R³ are independently selected from the group consisting of hydrogen, alkyl, haloalkyl, hydroxyalkyl, alkenyl, alkynyl, cycloalkyl, aryl, substituted aryl, heteroaryl, substituted heteroaryl, halo, hydroxy, alkoxy, and —O—R4 where R4 is a substituted or unsubstituted aryl;
R² is selected from the group consisting of hydrogen, alkyl, haloalkyl, hydroxyalkyl, alkenyl, alkynyl, cycloalkyl, aryl, substituted aryl, heteroaryl, substituted heteroaryl, halo, hydroxy, alkoxy, and —O—R⁴ where R⁴ is a substituted or unsubstituted aryl; and
Z is —CH²— or —C(O)—;

In another embodiment, the LpxC inhibitor can be selected from compounds having formula III:

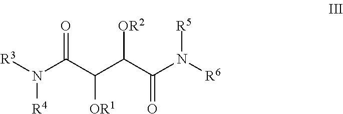

wherein:

(i) each of R¹ and R² independently is hydrogen or alkyl;

(ii) R³ and R⁴ taken together with the nitrogen to which they shown attached is heterocyclyl or heteroaryl, said heterocyclyl or heteroaryl having 1-3 heteroatoms including said nitrogen, said heterocyclyl or heteroaryl being optionally fused with aryl, heteroaryl, cycloalkyl, or heterocyclyl; wherein said heterocyclyl or heteroaryl comprising R³ and R⁴ is substituted with one or two substituents, each substituent being independently selected from the group consisting of aryl and alkynyl; wherein said aryl substituent is unsubstituted or is optionally substituted with one or two moieties selected independently from the group consisting of perhaloalkyl, halo, alkyl, alkoxy, cyano, perhaloalkoxy, and alkynyl moiety, wherein said alkynyl moiety is substituted with an aryl radical; wherein said alkynyl substituent is substituted with an aryl moiety, wherein said aryl moiety is unsubstituted or optionally substituted with one to three radicals selected from the group consisting of perhaloalkyl, halo, alkyl, alkoxy, cyano, and perhaloalkoxy; and

(iii) each of R⁵ and R⁶ is alkyl, or alternatively R⁵ and R⁶ taken together with the nitrogen to which they shown attached is heterocyclyl having 1-3 heteroatoms including said nitrogen; wherein said heterocyclyl comprising R⁵ and R⁶ is unsubstituted or optionally substituted with an aryl substituent; wherein said aryl substituent is unsubstituted or optionally substituted with one to three moieties independently selected from the group consisting of perhaloalkyl, halo, alkyl, alkoxy, cyano, and perhaloalkoxy;

with the proviso that the aryl substituent of said heterocyclyl or heteroaryl comprising R³ and R⁴ can be unsubstituted or optionally independently substituted with one to three moieties independently selected from the group consisting perhaloalkyl, halo, alkyl, alkoxy, cyano, and perhaloalkoxy only when R⁵ and R⁶ taken together with the nitrogen to which R⁵ and R⁶ are shown attached is heterocyclyl.

In another embodiment, the LpxC inhibitor can be selected from the group consisting of:

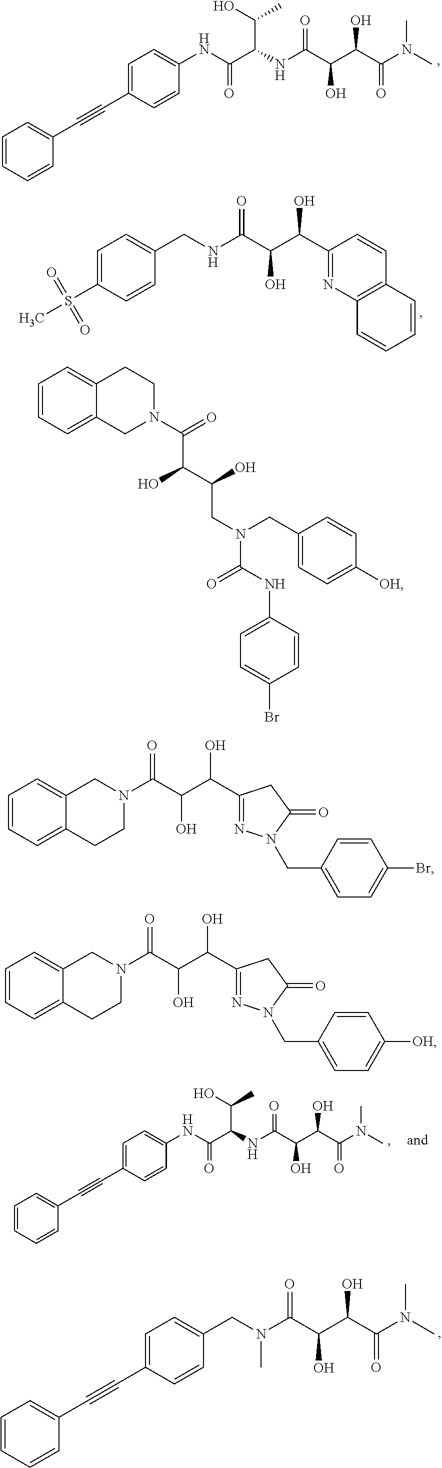

or a prodrug, solvate, ester or pharmaceutically acceptable salt thereof.

The above LpxC inhibitors may be made according to the methods disclosed in International PCT Application Publication Nos. 2004/007444, 2004/62601, 2007/064732 and 2008/154642, or by similar methods know to one skilled in the art.

### Pharmaceutical Compositions

Pharmaceutical compositions of the present invention comprise a therapeutically effective amount of a compound of the present invention formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials that can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and other animals orally, rectally, parenterally, intracisternally, intravaginally, intraperitoneally, topically (as by powders, ointments, or drops), bucally, or as an oral or nasal spray, or a liquid aerosol or dry powder formulation for inhalation.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution that, in turn, may depend upon crystal size and crystalline form. Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions that are compatible with body tissues.

Compositions for rectal or vaginal administration are preferably suppositories that can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and the like are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Compositions of the invention may also be formulated for delivery as a liquid aerosol or inhalable dry powder. Liquid aerosol formulations may be nebulized predominantly into particle sizes that can be delivered to the terminal and respiratory bronchioles where bacteria reside in patients with bronchial infections, such as chronic bronchitis and pneumonia. Pathogenic bacteria are commonly present throughout airways down to bronchi, bronchioli and lung parenchema, particularly in terminal and respiratory bronchioles. During exacerbation of infection, bacteria can also be present in alveoli. Liquid aerosol and inhalable dry powder formulations are preferably delivered throughout the endobronchial tree to the terminal bronchioles and eventually to the parenchymal tissue.

Aerosolized formulations of the invention may be delivered using an aerosol forming device, such as a jet, vibrating porous plate or ultrasonic nebulizer, preferably selected to allow the formation of aerosol particles having with a mass medium average diameter predominantly between 1 to 5 μm. Further, the formulation preferably has balanced osmolarity ionic strength and chloride concentration, and the smallest aerosolizable volume able to deliver effective dose of the compounds of the invention to the site of the infection. Additionally, the aerosolized formulation preferably does not impair negatively the functionality of the airways and does not cause undesirable side effects.

Aerosolization devices suitable for administration of aerosol formulations of the invention include, for example, jet, vibrating porous plate, ultrasonic nebulizers and energized dry powder inhalers, that are able to nebulize the formulation of the invention into aerosol particle size predominantly in the size range from 1-5 pm. Predominantly in this embodiment means that at least 70% but preferably more than 90% of all generated aerosol particles are 1 to 5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb and AeroDose vibrating porous plate nebulizers (AeroGen, Inc., Sunnyvale, Calif.), Sidestream7 nebulizers (Medic-Aid Ltd., West Sussex, England), Pari LC7 and Pari LC Star7 jet nebulizers (Pari Respiratory Equipment, Inc., Richmond, Va.), and Aerosonic (DeVilbiss Medizinische Produkte (Deutschland) GmbH, Heiden, Germany) and UltraAire7 (Omron Healthcare, Inc., Vernon Hills, Ill.) ultrasonic nebulizers.

Compounds of the invention may also be formulated for use as topical powders and sprays that can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present invention, bacterial infections are treated or prevented in a patient such as a human or lower mammal by administering to the patient a therapeutically effective amount of a compound of the invention, in such amounts and for such time as is necessary to achieve the desired result. By a “therapeutically effective amount” of a compound of the invention is meant a sufficient amount of the compound to treat bacterial infections, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The total daily dose of the compounds of this invention administered to a human or other mammal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 2000 mg of the compound(s) of this invention per day in single or multiple doses.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art.

A “kit” as used in the instant application includes a container for containing the pharmaceutical compositions and may also include divided containers such as a divided bottle or a divided foil packet. The container can be in any conventional shape or form as known in the art that is made of a pharmaceutically acceptable material, for example a paper or cardboard box, a glass or plastic bottle or jar, a resealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil that is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid, where the written memory aid is of the type containing information and/or instructions for the physician, pharmacist or other health care provider, or patient, e.g., in the form of numbers next to the tablets or capsules whereby the numbers correspond with the days of the regimen that the tablets or capsules so specified should be ingested or a card that contains the same type of information. Another example of such a memory aid is a calendar printed on the card e.g., as follows “First Week, Monday, Tuesday,” . . . etc . . . “Second Week, Monday, Tuesday, . . . ” etc. Other variations of memory aids will be readily apparent. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal that, for example, reads out the date that the last daily dose has been taken and/or reminds one when the next dose is to be taken.

The kits of the present invention may also include, in addition to LpxC inhibitors, one or more additional pharmaceutically active compounds. Preferably, the additional compound is another LpxC inhibitor or another compound useful to bacterial infections. The additional compounds may be administered in the same dosage form as the LpxC inhibitor or in different dosage forms. Likewise, the additional compounds can be administered at the same time as the LpxC inhibitor or at different times.

Compositions of the present compounds may also be used in combination with other known antibacterial agents of similar spectrum to (1) synergistically enhance treatment of severe Gram-negative infections covered by the spectrum of this compound or (2) add coverage in severe infections in which multiple organisms are suspected in which another agent of a different spectrum may be required in addition to this compound. Potential agents include members of the aminoglycosides, penicillins, cephalosporins, fluoroquinolones, macrolides, glycopeptides, lipopeptides and oxazolidinones. The treatment can involve administering a composition having both active agents or administration of the LpxC inhibitor followed by or preceded by administration of the additional active antibacterial agent.

### EXAMPLES

### Example 1

### Compromising the Outer Membrane of E. coli Enhances Activity of Many Antibacterial Agents

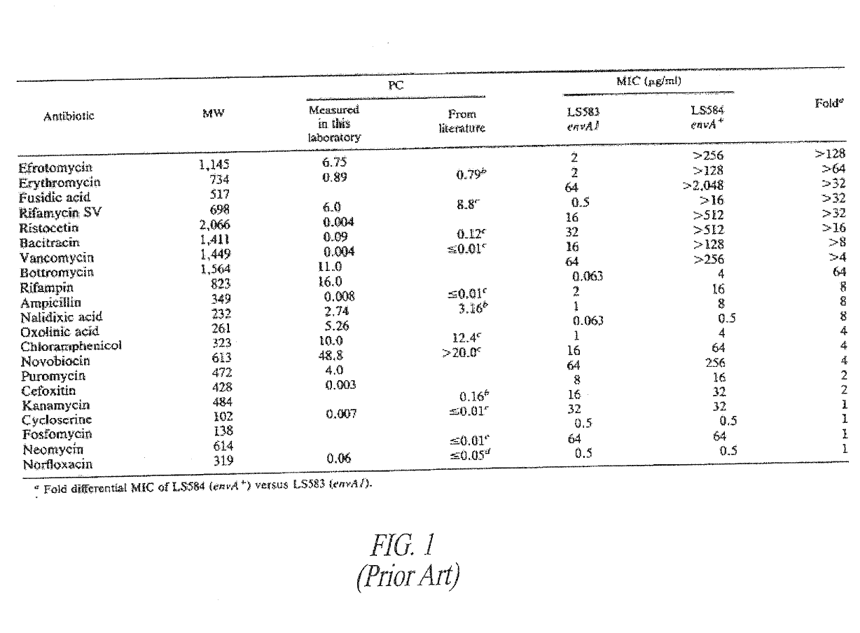

Young et al (“Leakage of Periplasmic Enzymes from envA1 Strains of Escherichia coli,” J. Bacteriol. 173(12):3609-14 (1991)) have previously reported that the envA1 mutation, a point mutation in the LpxC gene, results in a leaky outer membrane that is prone to release of periplasmic enzymes. FIG. 1, which replicates Table 3 of Young, et al. demonstrates that strains bearing the envA1 mutation are hypersensitive to a number of antibiotics. The enhanced sensitivity to these antibiotics is believed to be due to the fact that the intact outer membrane of wild type strains efficiently excludes these agents into the cell whereas the compromised outer membranes envA1 mutants allow such agents to much more readily diffuse into the cells to interact with their respective targets.

Similar experiments performed using isogenic envA1 (D22) and envA⁺ (D21) E. coli strains confirm an MIC shift of >8× for vancomycin, erythromycin and rifampin due to modification of envA (Table 1). In addition, these experiments demonstrate a shift of >8× in the MICs of daptomycin, teicoplanin, telavancin and oxacillin in this isogenic pair of strains.

TABLE 1

| Strain | LpxCi-4 | Daptomycin | Teicoplanin | Vancomycin | Telavancin | Oxacillin | Erythromycin | Rifampin |
|---|---|---|---|---|---|---|---|---|
| ATCC25922 | 0.015 | >256 | >256 | >256 | >128 | >128 | 64 | 16 |
| envA⁺ | 0.015 | 256 | >256 | >256 | >128 | >128 | 64 | 16 |
| (D21) | | | | | | | | |
| envA1 | ≦0.004 | 32 | 32 | 32 | 16 | 16 | 4 | 0.25 |
| (D21) | | | | | | | | |

| | | | | | | | |
|---|---|---|---|---|---|---|---|
| Fold decrease in | ≧4 | 8 | ≧8 | ≧8 | ≧8 | 16 | 64 |
| MIC | | | | | | | |
| (envA⁺/envA1) | | | | | | | |

### Example 2

### Synergy by EnvA Mutation is not Predictive of Checkerboard Synergy

Given that the envA1 mutation renders E. coli more sensitive to a broad range of antibiotics, it was hypothesized that some, or all, of these agents would demonstrate synergy when co-administered with an LpxC inhibitor. Partial or full inhibition of LpxC should compromise the outer membrane and thus allow these synergizing agents to enter the cells and exert an antibacterial effect at concentrations where cells untreated with an LpxC inhibitor are unaffected. As a first step, checkerboard synergy assays for a variety of antibacterial agents were perform with several LpxC inhibitors, on several species of gram negative bacteria.

Bacterial isolates were cultivated from −70° C. frozen stocks of the indicated strains by overnight passages at 35° C. in ambient air on Mueller-Hinton agar (Beckton Dickinson, Franklin Lakes, N.J.). Minimum Inhibitory Concentrations (MICs) were determined by the broth microdilution method in accordance with the Clinical and Laboratory Standards Institute (CLSI) guidelines. In brief, organism suspensions were adjusted to a 0.5 McFarland standard to yield a final inoculum between 3×10⁵ and 7×10⁵ colony-forming units (CFU)/mL. Drug dilutions and inocula were made in sterile, cation adjusted Mueller-Hinton Broth (Beckton Dickinson). An inoculum volume of 100 μL was added to wells containing 100 μL of broth with 2-fold serial dilutions of drug. All inoculated microdilution trays were incubated in ambient air at 35° C. for 18-24 hours. Following incubation, the lowest concentration of the drug that prevented visible growth (OD600 nm <0.05) was recorded as the MIC.

Standard checkerboard assays were performed with a combination of the indicated agents and LpxCi-4. Table 2 provides the FICI calculated according to standard techniques. While many of the agents, such as vancomycin, telavancin, teicoplanin, erythromycin, and rifampin, that showed sensitization in the envA1 mutants also gave FICI scores that indicate synergy (FICI ≦0.5), there were notable exceptions. Oxacillin and Daptomycin showed >8× shifts when the envA1 allele was present, yet they show no measurable synergy on K. pneumo. or P. aeruginosa. This indicates that a shift in MICs on the envA1 mutant of E. coli is not strongly predictive of seeing synergy by checkerboard in other Enterobacteriaceae or other gram-negative species such as Pseudomonas aeruginosa.

TABLE 2

| | FICI index | | | |
|---|---|---|---|---|
| | | | Esche- | |
| | Yersinia | Klebsiella | richia | Pseudomonas |
| | enterocolitica | pneumoniae | coli | aeruginosa |
| Vancomycin | ≦0.31 | ≦0.18 | ≦0.28 | ≦0.51 |
| Teicoplanin | ≦0.25 | ≦0.37 | n.d. | no effect |
| Daptomycin | n.d. | no effect | n.d. | no effect |
| Linezolid | n.d. | ≦0.5 | n.d. | no effect |
| Clindamycin | 0.13 | ≦0.5 | n.d. | no effect |
| Erythromycin | 0.09 | ≦0.13 | n.d. | ≦0.51 |
| Azithromycin | 0.09 | 0.14 | n.d. | 0.31 |
| Rifampin | 0.19 | ≦0.08 | n.d. | ≦1.0 |
| Oxacillin | n.d. | no effect | n.d. | no effect |
| Levofloxacin | 0.63 | 0.5 | n.d. | no effect |
| Ceftobioprole | 0.5 | 0.63 | n.d. | 0.5 |
| Cefotaxime | ≦0.5 | 0.5 | n.d. | ≦1.0 |
| Gentamicin | 0.5 | 0.75 | n.d. | 0.38 |
| Novobiocin | ≦0.63 | ≦0.38 | ≦0.25 | no effect |
| Telavancin | ≦0.5 | ≦0.31 | no | no effect |
| | | | effect | |

Since the level of synergy is sensitive to the relative concentrations of each component of the combination, Table 3 provides the synergy score for each antibacterial agent at ¼, 1/16 and 1/64 of its MIC as a single agent.

TABLE 3

| | | Synergy Score | | |
|---|---|---|---|---|
| | | LpxCi-1 | LpxCi-2 | LpxCi-3 |
| Azithromycin | ¼ MIC | 0.26 | 0.58 | 0.29 |
| | 1/16 MIC | 0.10 | 1.06 | 0.40 |
| | 1/64 MIC | 0.13 | 1.02 | 1.02 |
| Erythromycin | ¼ MIC | 0.36 | 1.25 | 0.58 |
| | 1/16 MIC | 0.17 | 1.06 | 0.40 |
| | 1/64 MIC | 0.35 | 1.02 | 0.35 |
| Linezolid | ¼ MIC | 0.54 | 1.25 | 0.58 |
| | 1/16 MIC | 0.46 | 1.06 | 1.06 |
| | 1/64 MIC | 1.03 | 1.02 | 1.02 |
| Vancomycin | ¼ MIC | 0.29 | 0.36 | 0.36 |
| | 1/16 MIC | 0.17 | 0.40 | 0.40 |
| | 1/64 MIC | 0.35 | 1.02 | 0.35 |
| Ciprofloxacin | ¼ MIC | 1.25 | 1.25 | 1.25 |
| | 1/16 MIC | 1.06 | 1.06 | 1.06 |
| | 1/64 MIC | 1.02 | 1.02 | 1.02 |
| Imipenem | ¼ MIC | 0.58 | 1.25 | 0.36 |
| | 1/16 MIC | 0.40 | 1.06 | 0.17 |
| | 1/64 MIC | .035 | 1.02 | 0.13 |

### Example 3

### Efficacy in Neutropenic Thigh Model

In vivo synergy of three LpxC inhibitors with vancomycin, rifampin, azithromycin, erythromycin, daptomycin or oxacillin was examined in the neutropenic thigh in vivo efficacy model. The model was run essentially as described by Craig and others (see Gudmundsson et al., “Murine Thigh Infection Model,” Handbook of Animal Models of Infection, M. A. Sande and O. Zak, Eds.; London: Academic Press, 1999, pp 137-144). Briefly, mice were rendered neutropenic prior to infection with 2 doses of cyclophosphamide, and then infected intramuscularly in the thigh with inocula of 10³-10⁵ CFU of either K. pneumo. (ATCC43816) or P. aeruginosa (ATCC27853). Antibiotics or vehicle alone as a negative control were administered twice at 2 hrs and 14 hrs post-infection. The animals were kept neutropenic for the duration of the experiment in order to minimize the effect of white blood cells on the infection such that the microbiological readout measures the in vivo interaction of drugs and bacteria. At 24 hrs post-infection, thighs were harvested, homogenized, and plated to measure the number of CFUs surviving per thigh. Thighs from a subset of animals were also harvested 2 hrs post-infection to record the CFUs present just prior to the first antibiotic treatment (pre-treatment). The static dose, defined as the dose required to result in a CFU load at 24 hours that is identical to that measured at 0 hours post infection, was calculated by standard methods in Prizm (GraphPad Software) from a dose response curve.

The purpose of these studies was to quantitatively assess whether the combination of these LpxC inhibitors with the respective candidate synergizing agents gave a greater reduction in counts in this in vivo efficacy model than does the sum of each agent alone.

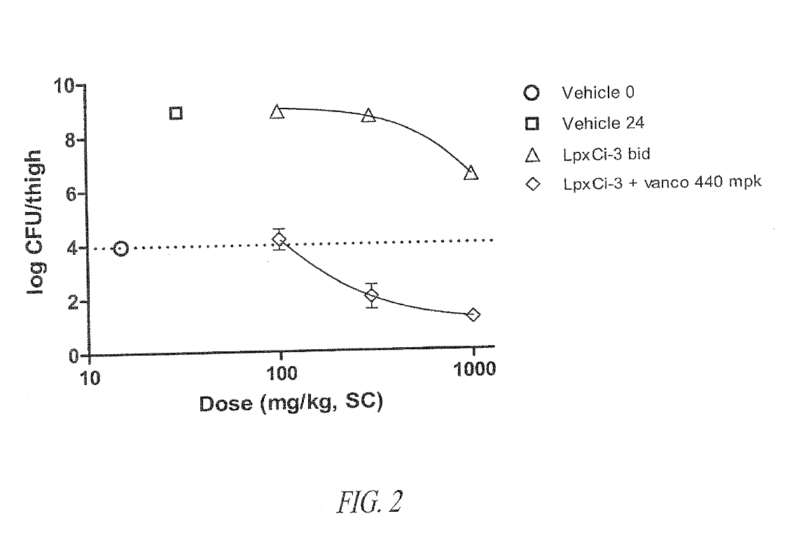

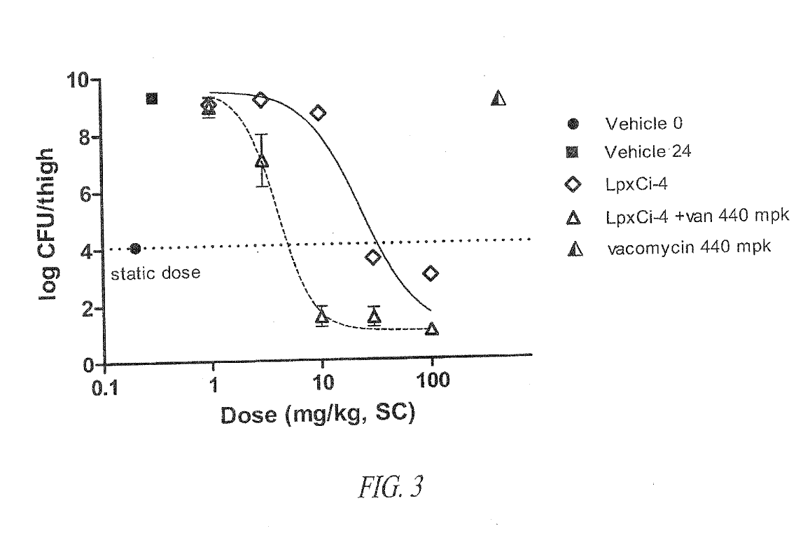

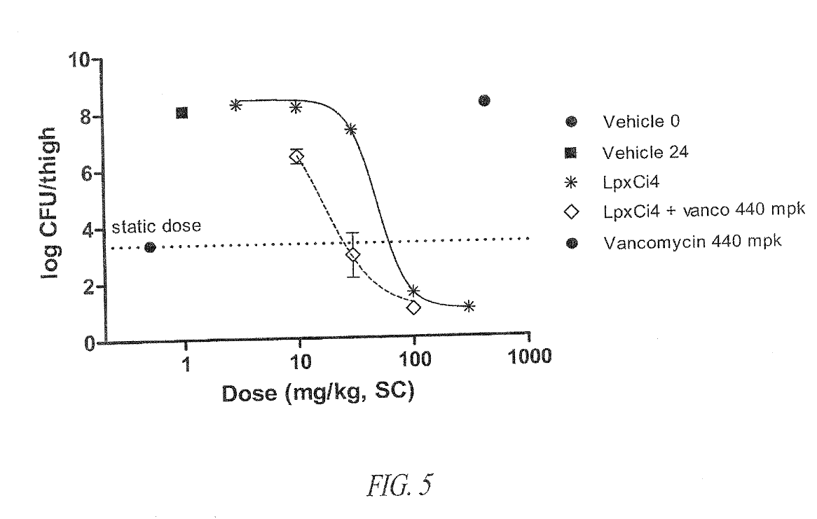

Vancomycin exhibits significant in vivo synergy with LpxC inhibitors for treatment of infections with ATCC43816. As indicated in FIGS. 2 and 3, treatment of infected mice with vancomycin alone at 440 mg/kg/day results in no significant reduction in CFU. However, when co-dosed with compounds LpxCi-3 or LpxCi-4 the static doses of the LpxC inhibitors is reduced by 7 to 9-fold (FIGS. 2 & 3; Table 4). Vancomycin also exhibits in vivo synergy with LpxCi-4 and LpxCi-6 for treatment of P. aeruginosa infections, with reductions of 1.8-2.4-fold in the respective static doses against this clinically important pathogen (FIGS. 5 & 6; Table 4). While it is formally possible that vancomycin might exert this synergistic effect by blocking the metabolism of LpxCi-4, thus increasing the in vivo exposure to LpxCi-4, as outlined in Example 7 below, the PK of LpxCi-4 is not significantly affected by co-dosing with vancomycin. Therefore this in vivo synergistic effect can be attributed to microbiological synergy in the in vivo setting.

TABLE 4

| | | | Static | | |
|---|---|---|---|---|---|
| | | Combination | Dose, | Static Dose, | Fold Shift |
| Strain | | agent (dose, | LpxC | LpxC in | Static |
| (ATCC#) | Cmpd | mg/kg/day) | cmpd | combination | Dose |
| 43816 | LpxCi-3 | Vanco | >1000 | 107 | >9.3x |
| 43816 | LpxCi-6 | Vanco | 240 | 80 | 3x |
| 43816 | LpxCi-4 | Vanco | 33 | 5 | 6.6x |
| 43816 | LpxCi-4 | Rifampin | 36 | 9 | 4x |
| 27853 | LpxCi-4 | Vanco | 60 | 25 | 2.4x |
| 27853 | LpxCi-6 | Vanco | 269 | 151 | 1.8x |
| 27853 | LpxCi-4 | Rifampin | 42 | 22 | 1.9x |
| 27853 | LpxCi-4 | Erythromycin | 50 | 40 | <1.5x |
| 27853 | LpxCi-4 | Daptomycin | 50 | 50 | — |
| 27853 | LpxCi-4 | Oxacillin | 50 | 50 | — |

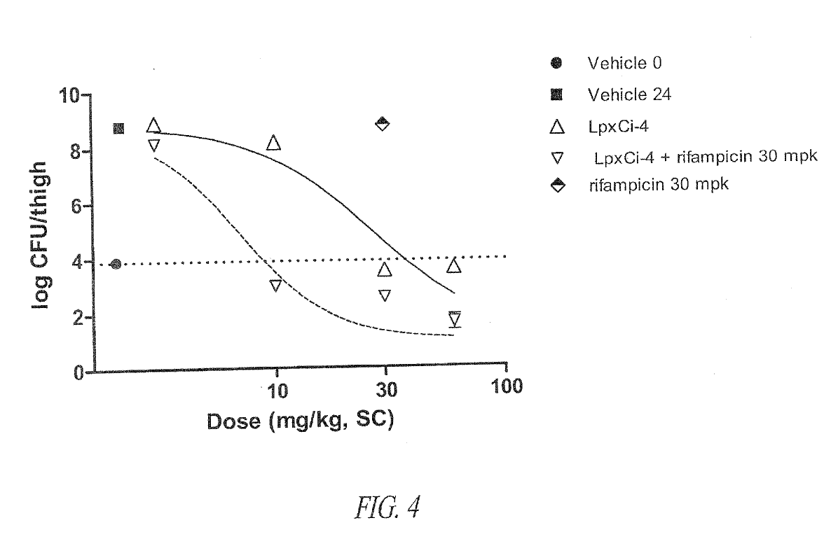

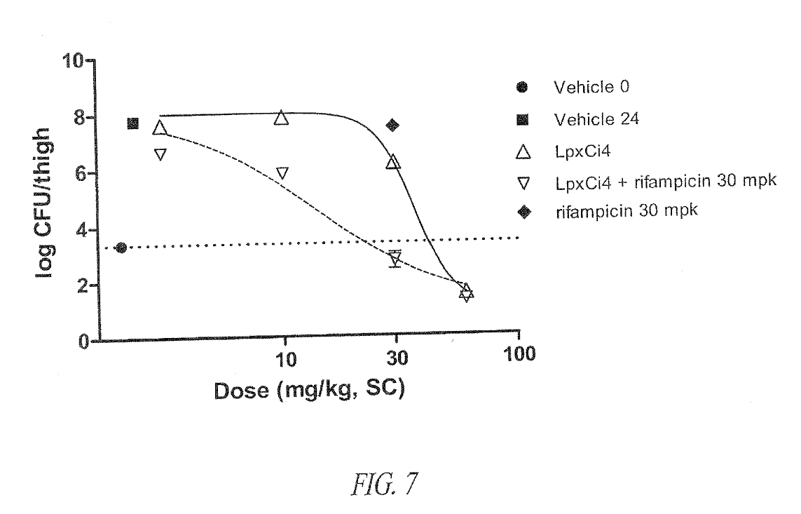

As indicated in FIGS. 4 and 7 and Table 4, rifampin exhibits significant in vivo synergy with LpxCi-4 for treatment of both K. pneumoniae (ATCC43816; 4-fold shift in static dose; Table 4) and P. aeruginosa (ATCC27853; 2-fold shift in static dose; Table 4).

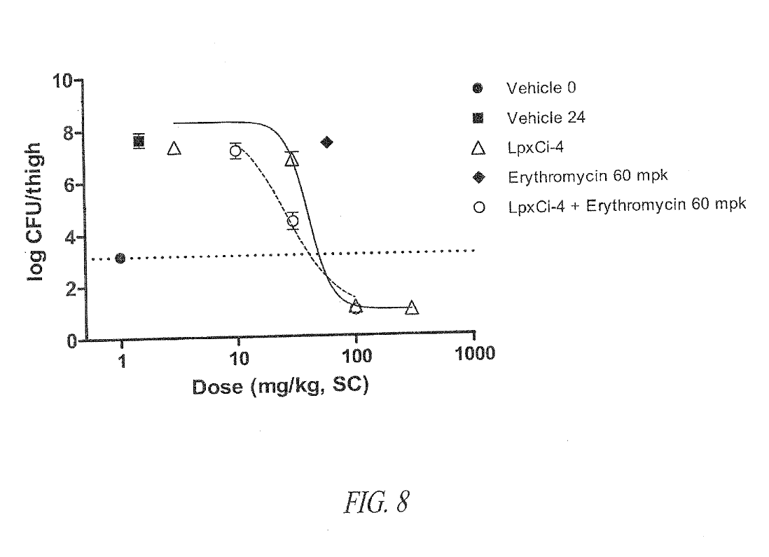

In contrast to these substantial shifts of the static doses of LpxC inhibitors on the P. aeruginosa strain ATCC27853 in the presence of vancomycin and rifampin, there is only a very modest shift in the dose response curve of LPxCi-4 when it is co-dosed with erythromycin at 60 mg/kg (FIG. 8). This is surprising in light of the fact that the synergy score of erythromycin on ATCC27853 (<0.05) is comparable to or less than the synergy scores of vancomycin (<0.5) or rifampin (<1.0), respectively, on this same strain. This data indicates that the presence of a strong synergy score (<0.5) in a checkerboard assay is not sufficient to predict strong in vivo synergy (shift of >2× in the static dose when agents combined relative to LpxCi-4 alone).

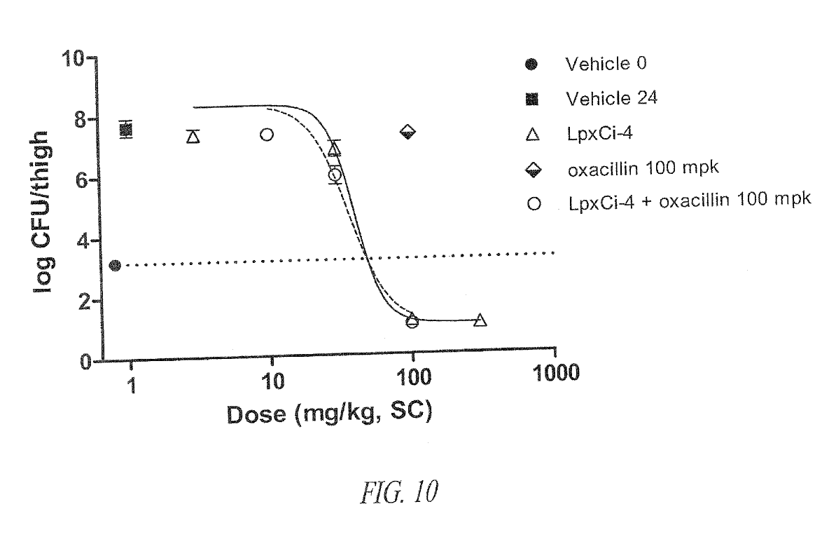

Oxacillin has a similarly very modest effect on the static dose of LpxCi-4 against ATCC27853 (FIG. 10). This is somewhat surprising in light of the fact that oxacillin's MIC is shifted >8-fold in strains with a compromised outer membrane (Table 1). This data indicates that sensitization of E. coli envA1 mutants to an agent of interest such as oxacillin is not sufficient to predict in vivo synergy between that agent and an LpxC inhibitor. The lack of in vivo synergy between oxacillin and LpxCi-4 is consistent with the lack of checkerboard synergy between these two agents (Table 2).

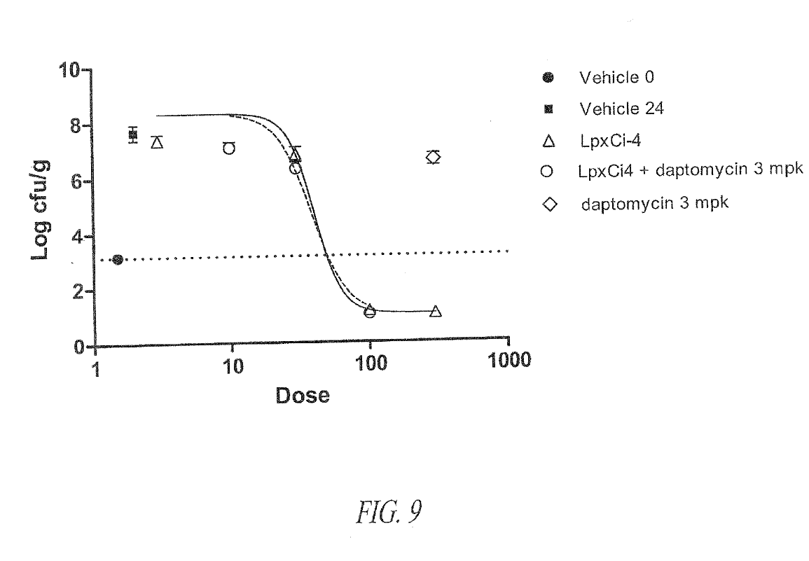

The MIC of daptomycin on the envA1 mutant is shifted 8× relative to the wild type strain (Table 1), thus indicating that daptomycin has intrinsic activity on this gram negative species and that there is an effect of the outer membrane on exclusion of this drug. However, daptomycin has no checkerboard synergy for either K. pneumo. or P. aeruginosa (Table 2) and no measurable in vivo synergy with 3936 for the treatment of ATCC27853 (FIG. 9; Table 4). This is another instance where sensitization of an envA1 strain is not sufficient to predict either in vitro (checkerboard) or in vivo synergy.

Table 5 summarizes in vivo synergy data for LpxCi-4 with several agents of interest. This table focuses on the sensitive part of the LpxCi-4 dose response curve (30 mg/kg) by examining the mean log CFU at 0 or 24 hours, as well as the log change in CFU at 24 hours relative to zero hours (“ΔLogCFU vs 24 hr”). Data is given for animals treated with vehicle, LpxCi-4 at 30 mg/kg, a second agent alone at the indicated doses or the combination of LpxCi-4 at 30 mg/kg plus the second agent at the indicated dose. The standard errors for each measurement are also given (n=5 animals/group). By comparing the observed drop in CFU of the combination with the sum of the drop in CFU associated with each of the two agents given separately, one can determine whether the combination is, in fact, synergistic in vivo. In vivo synergy occurs when the drop in CFU is greater than the sum of the drop in CFU of the individual agents. A striking example is that of rifampin, where rifampin alone at 30 mg/kg results in a drop of 0.28 log relative to vehicle and 3936 at 30 mg/kg results in a drop of 1.57 log CFU. If the agents did not interact and were purely additive, one would expect a drop of 1.85 log CFU. The observed drop with the combination is 5.05 log, indicating 3.2 logs greater effect than expected by a purely additive effect of the two agents. In contrast, oxacillin and daptomycin show very minimal (oxacillin) or no (daptomycin) evidence for in vivo synergy. We see a very pronounced increase in killing with azithromycin plus 3936 compared to 3936 alone (4.9 logs of additional killing). While we do not have in vivo data on azithromycin alone in this experiment, based on the fact that these agents are in the same class and that both azithromycin and erythromycin have MICs >16 μg/ml on ATCC278534, we expect that azithromycin would have very minimal killing in vivo as a single agent, as is the case with erythromycin, and we therefore take the 4.9 logs of additional killing by the combination relative to 3936 alone as evidence for in vivo synergy. This is consistent with the fact that we see a FICI score of 0.31 in the checkerboard assay of 3936+azithromycin on this same strain of P. aeruginosa (ATCC27853).

TABLE 5

| Time | Compound 1 | | Compound 2 | | Log CFU | | ΔLogCFU | ΔLogCFU vs |
|---|---|---|---|---|---|---|---|---|
| (hr) | Agent | Dose (**) | Agent | Dose (**) | Mean | SEM | vs 24 hr | “additive” |
| 0 | none | none | none | none | 3.32 | 0.04 | na | |
| 24 | none | none | none | none | 7.74 | 0.16 | na | |
| 24 | 3936 | 30 | none | none | 6.17 | 0.23 | −1.57 | |
| 24 | none | none | Rif. | 30 | 7.46 | 0.09 | −0.28 | |
| 24 | 3936 | 30 | Rif. | 30 | 2.69 | 0.28 | −5.05 | −3.2 |
| 24 | 3936 | 30 | Azith. | 50 | 2.85 | 0.24 | −4.89 | na |

Challenge strain: ATCC27853

| | | | | | | | | |
|---|---|---|---|---|---|---|---|---|
| 0 | none | none | none | none | 3.14 | 0.06 | na | |
| 24 | none | none | none | none | 7.64 | 0.29 | na | |
| 24 | 3936 | 30 | none | none | 6.83 | 0.26 | −0.81 | |
| 24 | none | none | Eryth. | 60 | 7.43 | 0.12 | −0.21 | |
| 24 | 3936 | 30 | Eryth. | 60 | 4.44 | 0.32 | −3.2 | −2.18 |
| 24 | none | none | Dapto. | 3 | 6.6 | 0.22 | −1.04 | |
| 24 | 3936 | 30 | Dapto. | 3 | 6.3 | 0.17 | −1.34 | +0.51 |
| 24 | none | none | Oxacil. | 100 | 7.29 | 0.18 | −0.35 | |
| 24 | 3936 | 30 | Oxacil. | 100 | 5.94 | 0.3 | −1.7 | −0.54 |

Challenge strain: ATCC27853
(*) ΔCFU is relative to vehicle CFU at 0 hours.
(**) mg/kg/day dosed b.i.d.

Taken together, these data show that rifampin and vancomycin demonstrate surprising in vivo synergy with multiple LpxC inhibitors on both K. pneumo. (ATCC43816) and P. aeruginosa (ATCC27853). Erythromycin, azithromycin and oxacillin demonstrate modest but potentially useful in vivo synergy.

### Example 4

### Suppression of Resistance

Vancomycin and LpxCi-5 were dissolved in 30% HPβCD (2-hydroxypropyl-β-cyclodextrin). Test substances were each administered subcutaneously (SC) singly or in combination to test animals at 2 hours and 14 hours after bacterial inoculation then bid daily for a total of 1, 2, 3 or 7 day(s). The dosing volume was 5 mL/kg.

Groups of 5 or 10 male specific-pathogen-free CD-1 (Crl.) mice weighing 24±2 g were used. Animals were immunosuppressed by two or three intraperitoneal injections of cyclophosphamide, the first at 150 mg/kg 4 days before infection (day −4) and the second or third at 100 mg/kg 1 day before or 3 days after infection (day −1 or +3). On day 0, animals were inoculated intramuscularly (0.1 mL/thigh) into the right thigh with a specific number (1.35×10⁷ CFU/mouse) of Klebsiella pneumoniae (ATCC 43816). Vehicle and test substances (singly or in combination) were each administered subcutaneously 2 hours and 14 hours after inoculation then bid for a total of 1, 2, 3 or 7 consecutive day(s). Groups at 24, 48, 72 hours and day 8 hours after treatment as the designated time of harvest, muscle of the right thigh was harvested from each of the survival test animals. An additional group with no treatment, at 2 hours after inoculation, the muscle of the right thigh was harvested and viable CFU on agar plates with or without drug were quantified by standard methods. Animals in groups designated for Day 8 harvest are 10 mice and daily tally of survivors were kept until harvest and recorded. A non-infected group was just treated with LpxCi-5 and kept out to Day 8 and then harvested. The removed muscle tissues were then homogenized in 3˜4 mL of PBS, pH 7.4, with a ceramic mortar. Homogenates of 0.1 mL were used for serial 10-fold dilutions and plated on Muller-Hinton Broth in 1.5% Bacto agar and plated in parallel with drug plates for CFU determination. The original inoculum was adjusted to 2×10⁸ CFU/0.1 mL and then plated onto the 3 different drug plates of LpxCi-5 for CFU count.

The immunity of animals treated with vehicle or vancomycin alone was suppressed by two intraperitoneal injections of cyclophosphamide, the first was at 150 mg/kg 4 days before infection (day −4) and the second was at 100 mg/kg 1 day before infection (day −1). On day 0, test compound and vehicle were each administered subcutaneously at 2 and 14 hrs after animals were inoculated intramuscularly (0.1 mL/thigh) with 1.35×10⁷ CFU/mouse of Klebsiella pneumoniae (ATCC 43816). All mice treated with vehicle or vancomycin alone died within 24 hours.

The immunity of animals treated with LpxCi-5 alone was suppressed by two or three intraperitoneal injections of cyclophosphamide, the first was at 150 mg/kg 4 days before infection (day −4); the second and the third was at 100 mg/kg 1 day before and three days after infection (day −1 and day +3). On day 0, test compound were each administered subcutaneously bid at 2 and 14 hrs after animals were inoculated intramuscularly (0.1 mL/thigh) with 1.35×10⁷ CFU/mouse of Klebsiella pneumoniae (ATCC 43816) for one, two, three or seven consecutive days. At 24, 48, 72 hours or at day 8 after treatment, muscle of the right thigh was harvested from each of the test animals for CFU determination in normal and drug plates. All animals were dead by Day 8.

The immunity of animals treated with both vancomycin and the LpxC inhibitor, LpxCi-5, was suppressed by two or three intraperitoneal injections of cyclophosphamide, the first was at 150 mg/kg 4 days before infection (day −4); the second and the third was at 100 mg/kg 1 day before and three days after infection (day −1 and day +3). On day 0, test compounds in combination were each administered subcutaneously bid at 2 and 14 hrs after animals were inoculated intramuscularly (0.1 mL/thigh) with 1.35×10⁷ CFU/mouse of Klebsiella pneumoniae (ATCC 43816) for one, two, three or seven consecutive days. At 24, 48, 72 hours or at day 8 after treatment, muscle of the right thigh was harvested from each of the test animals for CFU determination in normal and drug plates. Two animals treated with both vancomycin and an LpxC inhibitor died by Day 8. Table 6 provides the CFU determination on normal plates. Table 7 provides the CFU determination on drug plates.

LpxCi-5 (35 or 70 mg/kg), and Vancomycin at 25 mg/kg were administered subcutaneously (SC) singly or in combination at 2 and 14 hours post bacterial inoculation then bid daily for a total of 1, 2, 3 and 7 consecutive days for possible antimicrobial activity against a higher inoculum CFU of Klebsiella pneumoniae (ATCC 43816) in the neutropenic mouse infected thigh model in the designated time points. Quantitative counts of both total and resistant colony forming units (CFU) were obtained at the indicated time points. In this model an unusually high (10⁸ CFU/thigh) inoculum was used in order to ensure the existence of a population or resistant mutants at the time of infection. The model was run for 7 days so that this small initial population of resistant mutants was allowed to replicate under drug pressure. The goal was to identify a drug exposure that is sufficient to suppress the amplification of both sensitive and resistant mutants such animals survive the full course of therapy.

As indicated in Table 6, the inoculum was lethal to 100% of the animals in the vehicle or Vancomycin only treatment group at 24 hours, whereas LpxCi-5 at 35 mg/kg bid (total of 70 mg/kg/day) provides protection to the animals out to 72 hours.

TABLE 6

| | | | | Mean CFU/g thigh |
|---|---|---|---|---|
| Treatment | Dose | Route | Time | muscle (SEM) |
| Vehicle (30% | 5 mL/kg bid × 1 | SC | 24 hr | all animals died |
| CD) | | | | |
| Vancomycin | 25 mg/kg bid × 1 | SC | 24 hr | all animals died |
| LpxC-i | 35 mg/kg bid × 1 | SC | 24 hr | 3.81 × 10⁷ |
| | | | | (1.63 × 10⁷) |
| | 35 mg/kg bid × 2 | SC | 48 hr | 3.80 × 10⁸ |
| | | | | (1.57 × 10⁸) |
| | 35 mg/kg bid × 3 | SC | 72 hr | 1.48 × 10⁹ |
| | | | | (3.38 × 10⁸) |
| | 35 mg/kg bid × 7 | SC | Day 8 | all animals died |
| Vancomycin + | 25 mg/kg bid | SC | 24 hr | 1.13 × 10⁶ |
| LpxC-i | (vanco) × 1 | | | (2.61 × 10⁵) |
| | 35 mg/kg bid | | | |
| | (LpxC-i) × 1 | | | |
| | 25 mg/kg bid | SC | 48 hr | 2.53 × 10⁶ |
| | (vanco) × 2 | | | (1.34 × 10⁶) |
| | 35 mg/kg bid | | | |
| | (LpxC-i) × 2 | | | |
| | 25 mg/kg bid | SC | 72 hr | 1.61 × 10⁸ |
| | (vanco) × 3 | | | (9.82 × 10⁷) |
| | 35 mg/kg bid | | | |
| | (LpxC-i) × 3 | | | |
| | 25 mg/kg bid | SC | Day 8 | 5.53 × 10⁸ |
| | (vanco) × 7 | | | (5.53 × 10⁸) |
| | 35 mg/kg bid | | | |
| | (LpxC-i) × 7 | | | |

+: Too many colonies to permit accurate counting.

However, the total burden of CFU increases from ˜10⁷ CFU/thigh to >10⁹ CFU/thigh by 72 hours, and there is a large population (>10³ CFU/thigh) of resistant CFU present at 24, 48 and 72 hours in these animals. In contrast, the combination of LpxCi-5 at 35 mg/kg bid (70 mg/kg/day) and Vancomycin at 25 mg/kg bid (50 mg/kg/day) resulted in significantly lower total and resistant colony counts at 24, 48 and 72 hours compared to LpxCi-5 alone (Table 7). Three of the five animals treated with the combination survived to 8 days, compared to zero animals treated with either drug alone. Two of the surviving animals had <100 resistant CFU in the 10⁰ dilution, indicating that the combination of LpxCi-5 plus Vancomycin results in a significant suppression of amplification of resistant mutants compared to LpxCi-5 alone (Table 7).

TABLE 7

| | | | | | CFU/Drug | |
|---|---|---|---|---|---|---|
| | | | | | Plate - | |
| | | | | | Dilution | |
| Treatment | Dose | Route | Time | N | 10⁰ | 10⁻¹ |
| LpxC-i | 35 mg/kg bid × 1 | SC | 24 hr | 1 | + | + |
| | | | | 2 | + | + |
| | | | | 3 | + | + |
| | | | | 4 | + | 780 |
| | | | | 5 | + | + |
| | 35 mg/kg bid × 2 | SC | 48 hr | 1 | + | + |
| | | | | 2 | + | + |
| | | | | 3 | + | + |
| | | | | 4 | + | + |
| | | | | 5 | + | + |
| | 35 mg/kg bid × 3 | SC | 72 hr | 1 | + | + |
| | | | | 2 | + | + |
| | | | | 3 | + | + |
| | | | | 4 | + | + |
| | | | | 5 | + | + |
| Vancomycin + | 25 mg/kg bid | SC | 24 hr | 1 | 884 | 72 |
| LpxC-i | (vanco) × 1 | | | 2 | + | 6 |
| | 35 mg/kg bid | | | 3 | + | 9 |
| | (LpxC-i) × 1 | | | 4 | + | 0 |
| | | | | 5 | + | 0 |
| | 25 mg/kg bid | SC | 48 hr | 1 | + | 45 |
| | (vanco) × 2 | | | 2 | + | 114 |
| | 35 mg/kg bid | | | 3 | + | 0 |
| | (LpxC-i) × 2 | | | 4 | + | 15 |
| | | | | 5 | + | 100 |
| | 25 mg/kg bid | SC | 72 hr | 1 | + | 222 |
| | (vanco) × 3 | | | 2 | 0 | 0 |
| | 35 mg/kg bid | | | 3 | + | + |
| | (LpxC-i) × 3 | | | 4 | + | + |
| | | | | 5 | + | 271 |
| | 25 mg/kg bid | SC | Day 8 | 1 | 1 | 0 |
| | (vanco) × 7 | | | 2 | 68 | 0 |
| | 35 mg/kg bid | | | 3 | + | + |
| | (LpxC-i) × 7 | | | 4 | dead | |
| | | | | 5 | dead | |

The results indicate that LpxCi-5 is synergistic with Vancomycin in vivo and that the combination of these two agents demonstrably suppresses the emergence of resistant mutants in vivo.

### Example 5

### Presence of Vancomycin does not Alter Pharmacokinetics of LpxC Inhibitor

One possible interpretation of the in vivo synergy data is that the presence of Vancomycin simply blocks the clearance of LpxC inhibitors, thus increasing the amount of drug on board (increase in AUC). This would result in a lower static dose for the LpxC inhibitor and apparent synergy. In order to address this, pharmacokinetic studies of LpxCi-4 either alone or in the presence of vancomycin at 220 mg/kg were performed. As indicated in Table 8, the AUC of LpxCi-4 is essentially identical in the presence or absence of vancomycin. Thus, the in vivo synergy is not due to a pharmacokinetic effect and the in vivo data is consistent with the hypothesis that the increased in vivo potency of LpxCi-4 in the presence of vancomycin is indeed due to microbiological synergy. The half-life (HL_Lambda_z), time to maximum concentration (Tmax), maximal concentration (Cmax), and area under the concentration-time curve (AUC) for LpxCi-4 are provided.

TABLE 8

| | | LpxCi-4 (10 mg/kg) + | |
|---|---|---|---|
| parameter | units | vancomycin (220 mg/kg) | LpxCi-4 (10 mg/kg) |
| Half life | hr | 3.27 (±error) | 2.63 (±error) |
| Tmax | hr | 0.25 (±error) | 0.50 (±error) |
| Cmax | μg/ml | 2.63 (±error) | 2.20 (±error) |
| AUC | hr * μg/ml | 6.87 (±error) | 6.03 (±error) |

Furthermore, while particular embodiments of the present invention have been shown and described herein for purposes of illustration, it will be understood, of course, that the invention is not limited thereto since modifications may be made by persons skilled in the art, particularly in light of the foregoing teachings, without deviating from the spirit and scope of the invention. Accordingly, the invention is not limited except as by the appended claims.

All of the U.S. patents, U.S. patent application publications, U.S. patent applications, foreign patents, foreign patent applications and non-patent publications referred to in this specification are incorporated herein by reference, in their entirety to the extent not inconsistent with the present description.

What is claimed is:

1. A pharmaceutical composition comprising a synergistic combination of an antibacterial agent and an inhibitor of LpxC.

2. The pharmaceutical composition of claim 1, wherein the antibacterial agent is selected from the group consisting of vancomycin, linezolid, azithromycin, imipenem, teicoplanin, daptomycin, clindamycin, rifampin, cefotaxime, gentamicin, novobiocin, and telavancin.

3. The pharmaceutical composition of claim 2, wherein the antibacterial agent is vancomycin or rifampin.

4. The pharmaceutical composition of claim 1, wherein the synergistic combination demonstrates in vivo synergy.

5. The pharmaceutical composition of claim 1, wherein the LpxC inhibitor is a compound of formula (I), or a stereoisomer, pharmaceutically acceptable salt, ester, or prodrug thereof: 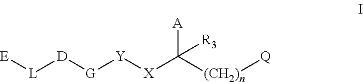
or a stereoisomer, pharmaceutically acceptable salt, ester, or prodrug thereof, wherein
E is absent or selected from the group consisting of
(1) H,
(2) substituted or unsubstituted C₁-C₆-alkyl,
(3) substituted or unsubstituted C₂-C₆-alkenyl,
(4) substituted or unsubstituted C₂-C₆-alkynyl,
(5) substituted or unsubstituted aryl,
(6) substituted or unsubstituted heterocyclyl, and
(7) substituted or unsubstituted heteroaryl;
L is absent or selected from the group consisting of
(1) substituted or unsubstituted C₁-C₆-alkyl,
(2) —(NH)₀₋₁—(CH₂)ⱼ—NR^{3L}—(CH₂)ₖ—,
(3) —(NH)₀₋₁—C(R^{1L},R^{2L})—NR^{3L}—C(R^{1L},R^{2L})—,
(4) —C(R^{1L},R^{2L})—O—C(R^{1L},R^{2L})—,
(5) —(CH₂)ⱼ—NR^{3L}—C(R^{1L},R^{2L})—CONH—(CH₂)ₖ—,
(6) —CO—C(R^{1L},R^{2L})—NHCO—,
(7) —CONH—,
(8) —NHCO—,
wherein R^{1L}, R^{2L}, and R^{3L} are independently selected from the group consisting of (a) H, (b) substituted or unsubstituted C₁-C₆-alkyl, (c) C₁-C₆-alkyl substituted with aryl, (d) C₁-C₆-alkyl substituted with heterocyclyl, and (e) C₁-C₆-alkyl substituted with heteroaryl, or R^{1L} and R^{3L}, together with the atoms to which they are attached can form a substituted or unsubstituted heterocyclic ring, having from 3 to 8 ring atoms, wherein 1-2 ring atoms of the heterocyclic ring system are selected from N, O and S;
j is an integer of 0-4;
k is an integer of 0-4;
D is absent or selected from the group consisting of
(1) substituted or unsubstituted C₃-C₈-cycloalkyl,
(2) substituted or unsubstituted aryl,
(3) substituted or unsubstituted heterocyclyl, and
(4) substituted or unsubstituted heteroaryl;
G is absent or selected from the group consisting of
(1) —(CH₂)ᵢ—O—(CH₂)ᵢ—,
(2) —(CH₂)ᵢ—S—(CH₂)ᵢ—,
(3) —(CH₂)ᵢ—NR^{g}—(CH₂)ᵢ—,
(4) —C(═O)—,
(5) —NHC(═O)—,
(6) —C(═O)NH—,
(7) —(CH₂)ᵢNHCH₂C(═O)NH—,
(8) —C≡C—,
(9) —C≡C—C≡C—, and
(10) —C═C—;
wherein R^{g} is H or substituted or unsubstituted C₁-C₆-alkyl;
i is an integer of 0-4;
Y is selected from the group consisting of
(1) substituted or unsubstituted C₃-C₈-cycloalkyl,
(2) substituted or unsubstituted aryl,
(3) substituted or unsubstituted heterocyclyl, and
(4) substituted or unsubstituted heteroaryl;
X is selected from the group consisting of
(1) —(C═O)—,
(2) —C₁-C₆-alkyl-(C═O)C—,
(3) —C₂-C₆-alkenyl-(C═O)—,
(4) —C₂-C₆-alkynyl-(C═O)—, and
(5) —CH₂—;
or when B is absent, X and A, together with the atoms to which they are attached can form a heterocyclic ring, having from 5 to 8 ring atoms, wherein 1-2 ring atoms of the heterocyclic ring system are selected from N, O and S;
B is absent or 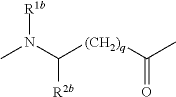
wherein R^{1b} and R^{2b}, are independently selected from the group consisting of (a) H, (b) substituted or unsubstituted C₁-C₆-alkyl, (c) substituted or unsubstituted C₂-C₆-alkenyl, (d) substituted or unsubstituted C₂-C₆-alkynyl, (e) substituted or unsubstituted aryl, (f) substituted or unsubstituted heterocyclyl, (g) substituted or unsubstituted heteroaryl, (h) C₁-C₆-alkyl substituted with aryl, (i) C₁-C₆-alkyl substituted with heterocyclyl, and (j) C₁-C₆-alkyl substituted with heteroaryl,
or R^{1b} and R^{2b}, together with the atoms to which they are attached can form a substituted or unsubstituted heterocyclic ring, having from 5 to 8 ring atoms, wherein 1-2 ring atoms of the heterocyclic ring system are selected from N, O and S;
q is an integer of 0-4;
R₃ is H or substituted or unsubstituted C₁-C₆-alkyl,
or R₃ and A, together with the atoms to which they are attached can form a substituted or unsubstituted 3-10 membered cycloalkyl or a heterocyclic ring system, wherein the heterocyclic ring system may have from 3 to 10 ring atoms, with 1 to 2 rings being in the ring system and contain from 1-4 heteroatoms selected from N, O and S;
R⁴ is H or substituted or unsubstituted C₁-C₆-alkyl,
or R₄ and A, together with the atoms to which they are attached can form a substituted or unsubstituted heterocyclic ring, having from 3 to 8 ring atoms, wherein 1-2 ring atoms of the heterocyclic ring system are selected from N, O and S; n is an integer of 0-6;
A is selected from the group consisting of
(1) H,
(2) —(CH₂)ᵣC(R^{1a},R^{2a})(CH₂)OR^{3a},
(3) —(CH₂)ᵣC(R^{1a},R^{2a})N(R^{4a},R^{5a}),
(4) —(CH₂)ᵣC(R^{1a},R^{2a})N(R^{4a})COR^{3a},
(5) —(CH₂)ᵣC(R^{1a},R^{2a})NHCON(R^{4a},R^{5a}),
(6) —(CH₂)ᵣC(R^{1a},R^{2a})NHC(═NH)N(R^{4a},R^{5a}),
(7) —CH(R^{1a},R^{2a}),
(8) —C≡CH,
(9) —(CH₂)ᵣC(R^{1a},R^{2a})CN,
(10) —(CH₂)ᵣC(R^{1a},R^{2a})CO₂R^{3a}, and
(11) —(CH₂)ᵣC(R^{1a},R^{2a})CON(R^{4a},R^{5a}),
wherein R^{1a}, R^{2a}, R^{3a}, R^{4a}, and R^{5a} are independently selected from the group consisting of (a) H, (b) substituted or unsubstituted C₁-C₆-alkyl, (c) substituted or unsubstituted aryl, (d) substituted or unsubstituted heterocyclyl, (e) substituted or unsubstituted heteroaryl, (f) C₁-C₆-alkyl substituted with aryl, (g) C₁-C₆-alkyl substituted with heterocyclyl, and (h) C₁-C₆-alkyl substituted with heteroaryl, or R^{4a} and R^{5a} together with the N atom to which they are attached can form a substituted or unsubstituted heterocyclic ring, having from 5 to 8 ring atoms, wherein 1-2 ring atoms of the heterocyclic ring system are selected from N, O and S;
r is an integer of 0-4;
s is an integer of 0-4;
Q is absent or selected from the group consisting of
(1) —C(═O)N(R₁,R₂),
(2) —NHC(═O)N(R₁,R₂),
(3) —N(OH)C(═O)N(R₁,R₂),
(4) —CH(OH)C(═O)N(R₁,R₂),
(5) —CH[N(R^{2q},R^{3q})]C(═O)N(R₁,R₂),
(6) —CHR^{1q}C(═O)N(R₁,R₂),
(7) —CO₂H,
(8) —C(═O)NHSO₂R^{4q},
(9) —SO₂NH₂,
(10) —N(OH)C(═O)R^{1q},
(11) —N(OH)SO₂R^{4q},
(12) —NHSO₂R^{4q},
(13) —SH,
(14) —CH(SH)(CH₂)₀₋₁C(═O)N(R₁,R₂),
(15) —CH(SH)(CH₂)₀₋₁CO₂H,
(16) —CH(OH)(CH₂)₀₋₁CO₂H,
(17) —CH(SH)CH₂CO₂R^{1q},
(18) —CH(OH)(CH₂)SO₂NH₂,
(19) —CH(CH₂SH)NHCOR^{1q},
(20) —H(CH₂SH)NHSO₂R^{4q},
(21) —CH(CH₂SR^{5q})CO₂H,
(22) —CH(CH₂SH)NHSO₂NH₂,
(23) —CH(CH₂OH)CO₂H,
(24) —H(CH₂OH)NHSO₂NH₂,
(25) —C(═O)CH₂CO₂H,
(26) —C(═O)(CH₂)₀₋₁CONH₂,
(27) —OSO₂NHR^{5q},
(28) —SO₂NHNH₂,
(29) —P(═O)(OH)₂, 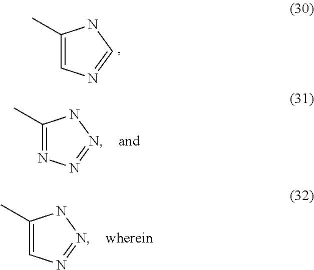
R₁ is selected from the group consisting of (1) H, (2) —OH, (3) —OC₁₋₆-alkyl, (4) —N(R^{2q},R^{3q}), and (5) substituted or unsubstituted C₁₋₆-alkyl;
R₂ is selected from the group consisting of
(1) H,
(2) substituted or unsubstituted C₁-C₆-alkyl,
(3) substituted or unsubstituted C₂-C₆-alkenyl,
(4) substituted or unsubstituted C₂-C₆-alkenyl,
(5) substituted or unsubstituted aryl,
(6) substituted or unsubstituted heterocyclyl,
(7) substituted or unsubstituted heteroaryl,
(8) C₁-C₆-alkyl substituted with aryl,
(9) C₁-C₆-alkyl substituted with heterocyclyl, and
(10) C₁-C₆-alkyl substituted with heteroaryl,
or R¹ and R², together with the N atom to which they are attached can form a substituted or unsubstituted heterocyclic ring, having from 3 to 10 ring atoms, wherein 1-4 ring atoms of the heterocyclic ring system are selected from N, O and S, or R² and R⁴, together with the N atoms to which they are attached can form a substituted or unsubstituted heterocyclic ring, having from 3 to 10 ring atoms, wherein 1-4 ring atoms of the heterocyclic ring system are selected from N, O and S;
R^{1q}, R^{2q}, R^{3q}, R^{4q}, and R^{5q} are selected from H or C₁-C₆ alkyl, wherein B is absent, or E, L, G, and B are absent, or E, L, and G are absent, or E, L, and B are absent, or E, L, D, G, and B are absent.

6. The compound of claim 1, wherein the LpxC inhibitor is a compound of formula (I), or a stereoisomer, pharmaceutically acceptable salt, ester, or prodrug thereof: 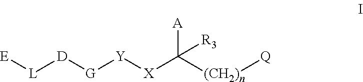
wherein:
E is selected from the group consisting of:
(1) H,
(2) substituted or unsubstituted C₁-C₆-alkyl,
(3) substituted or unsubstituted C₂-C₆-alkenyl,
(4) substituted or unsubstituted C₂-C₆-alkynyl,
(5) substituted or unsubstituted C₃-C₁₀-cycloalkyl,
(6) substituted or unsubstituted aryl,
(7) substituted or unsubstituted heterocyclyl, and
(8) substituted or unsubstituted heteroaryl;
L is absent or selected from the group consisting of:
(1) substituted or unsubstituted C₁-C₆-alkyl,
(2) —(NR^{3L})₀₋₁—(CH₂)₀₋₄—NR^{3L}—(CH₂)₀₋₄—,
(3) —(NR^{3L})₀₋₁—C(R^{1L},R^{2L})—NR^{3L}—C(R^{1L},R^{2L})—,
(4) —C(R^{1L},R^{2L})—O—C(R^{1L},R^{2L})—,
(5) —(CH₂)₀₋₄—NR^{3L}—C(R^{1L},R^{2L})—CONH—(CH₂)₀₋₄—,
(6) —CO—C(R^{1L},R^{2L})—NHCO—,
(7) —CONR^{3L}—,
(8) —NR^{3L}CO—,
(9) —NR^{3L}—,
(10) —SO₂NR^{3L}—,
(11) —NR^{3L}—C(═O)—NR^{3L}—,
(12) substituted or unsubstituted C₃-C₁₀-cycloalkyl,
(13) substituted or unsubstituted aryl,
(14) substituted or unsubstituted heterocyclyl, and
(15) substituted or unsubstituted heteroaryl,
wherein:
each R^{1L}, R^{2L}, and R^{3L} is independently selected from the group consisting of:
(a) H,
(b) substituted or unsubstituted C₁-C₆-alkyl,
(c) C₁-C₆-alkyl substituted with aryl,
(d) C₁-C₆-alkyl substituted with heterocyclyl, and
(e) C₁-C₆-alkyl substituted with heteroaryl,
or R^{1L} and R^{3L}, together with the atoms to which they are attached can form a substituted or unsubstituted heterocyclic ring, having from 3 to 8 ring atoms, wherein 1-2 ring atoms of the heterocyclic ring are selected from N, O and S;
D is absent or selected from the group consisting of:
(1) substituted or unsubstituted C₃-C₁₀-cycloalkyl,
(2) substituted or unsubstituted aryl,
(3) substituted or unsubstituted heterocyclyl, and
(4) substituted or unsubstituted heteroaryl;
G is selected from the group consisting of:
(1) —NR^{1G}C(═O)—,
(2) —C(═O)NR^{1G}—,
(3) —(CH₂)₀₋₄NHCH₂C(═O)NR^{1G}—,
(4) —CR^{2G}═CR^{2G}—,
(5) —S(═O)—,
(6) —SO₂—,
(7) —C(R^{3G})₂—S(═O)—,
(8) —S(═O)—C(R^{3G})₂—,
(9) —C(R^{3G})₂—SO₂—,
(10) —SO₂—C(R^{3G})₂—,
(11) —CR^{3G}═CR^{3G}—CR^{3G}═CR^{3G}—,
(12) —C(R^{3G})₂—,
(13) —CR^{3G}═CR^{3G}—C≡C—,
(14) —C≡C—CR^{3G}═CR^{3G}—,
(15) —C(═O)—C≡C—,
(16) —C≡C—C(═O)—,
(17) substituted or unsubstituted C₃-C₁₀-cycloalkyl,
(18) substituted or unsubstituted aryl,
(19) substituted or unsubstituted heterocyclyl, and
(20) substituted or unsubstituted heteroaryl,
wherein:
R^{1G} is substituted or unsubstituted C₁-C₆-alkyl;
each R^{2G} is independently selected from the group consisting of H, a halogen atom, and substituted or unsubstituted C₁-C₆-alkyl, and at least one R^{2G} is not H; and
R^{3G} is selected from the group consisting of H, a halogen atom, and substituted or unsubstituted C₁-C₆-alkyl;
Y is absent or selected from the group consisting of:
(1) substituted or unsubstituted C₃-C₁₀-cycloalkyl,
(2) substituted or unsubstituted aryl,
(3) substituted or unsubstituted heterocyclyl, and
(4) substituted or unsubstituted heteroaryl;
X is selected from the group consisting of:
(1) —(C═O)NR₄—,
(2) —C₁-C₆-alkyl-(C═O)NR₄—,
(3) —C₂-C₆-alkenyl-(C═O)NR₄—,
(4) —C₂-C₆-alkynyl-(C═O)NR₄—,
(5) —CH₂N
(6) —SO₂NR₄—,
(7) —S(═O)NR₄—,
(8) —NR₄C(═O)—, and
(9) —NR₄—,
or X and A, together with the atoms to which they are attached can form a heterocyclic ring, having from 5 to 8 ring atoms, wherein 1-2 ring atoms of the heterocyclic ring are selected from N, O and S,
or when Y is a bicyclic substituted or unsubstituted heterocyclyl or heteroaryl, then X is absent;
R₃ is H or substituted or unsubstituted C₁-C₆-alkyl, or R₃ and A, together with the atom to which they are attached can form a substituted or unsubstituted 3-10 membered cycloalkyl or a heterocyclic ring, having from 3 to 10 ring atoms, wherein 1-4 ring atoms of the heterocyclic ring are selected from N, O and S;
R₄ is (1) H or substituted or unsubstituted C₁-C₆-alkyl, or (2) R₄ and A, together with the atoms to which they are attached can form a substituted or unsubstituted heterocyclic ring, having from 3 to 8 ring atoms, wherein 1-2 ring atoms of the heterocyclic ring are selected from N, O and S, or (3) R₄ and Y, together with the atoms to which they are attached, form a bicyclic substituted or unsubstituted heterocyclyl or heteroaryl;
n is an integer from 0-6;
A is selected from the group consisting of:
(1) H,
(2) —(CH₂)₀₋₄C(R^{1a},R^{2a})(CH₂)₀₋₄OR^{3a},
(3) —(CH₂)₀₋₄C(R^{1a},R^{2a})N(R^{4a},R^{5a}),
(4) —(CH₂)₀₋₄C(R^{1a},R^{2a})N(R^{4a})COR^{3a},
(5) —(CH₂)₀₋₄C(R^{1a},R^{2a})NHCON(R^{4a},R^{5a}),
(6) —(CH₂)₀₋₄C(R^{1a},R^{2a})NHC(═NH)N(R^{4a},R^{5a}),
(7) —CH(R^{1a},R^{2a}),
(8) —C≡CH,
(9) —(CH₂)₀₋₄C(R^{1a},R^{2a})CN,
(10) —(CH₂)₀₋₄C(R^{1a},R^{2a})CO₂R^{3a},
(11) —(CH₂)₀₋₄C(R^{1a},R^{2a})CON(R^{4a},R^{5a}),
(12) substituted or unsubstituted C₃-C₁₀-cycloalkyl,
(13) substituted or unsubstituted aryl,
(14) substituted or unsubstituted heterocyclyl, and
(15) substituted or unsubstituted heteroaryl,
wherein:
each R^{1a}, R^{2a}, R^{3a}, R^{4a}, and R^{5a} is independently selected from the group consisting of:
(a) H,
(b) a halogen atom,
(c) substituted or unsubstituted C₁-C₆-alkyl,
(d) substituted or unsubstituted aryl,
(e) substituted or unsubstituted heterocyclyl, and
(f) substituted or unsubstituted heteroaryl,
or R^{4a} and R^{5a} together with the N atom to which they are attached can form a substituted or unsubstituted heterocyclic ring, having from 5 to 8 ring atoms, wherein 1-2 ring atoms of the heterocyclic ring are selected from N, O and S;
Q is absent or selected from the group consisting of:
(1) —C(═O)N(R₁,R₂),
(2) —NHC(═O)N(R₁,R₂),
(3) —N(OH)C(═O)N(R₁,R₂),
(4) —CH(OH)C(═O)N(R₁,R₂),
(5) —CH[N(R^{2q},R^{3q})]C(═O)N(R₁,R₂),
(6) —CHR^{1q}C(═O)N(R₁,R₂),
(7) —CO₂H,
(8) —C(═O)NHSO₂R^{4q},
(9) —SO₂NH₂,
(10) —N(OH)C(═O)R^{1q},
(11) —N(OH)SO₂R^{4q},
(12) —NHSO₂R^{4q},
(13) —SH,
(14) —CH(SH)(CH₂)₀₋₁C(═O)N(R₁,R₂),
(15) —CH(SH)(CH₂)₀₋₁CO₂H,
(16) —CH(OH)(CH₂)₀₋₁CO₂H,
(17) —CH(SH)CH₂CO₂R^{1q},
(18) —CH(OH)(CH₂)SO₂NH₂,
(19) —CH(CH₂SH)NHCOR^{1q},
(20) —CH(CH₂SH)NHSO₂R^{4q},
(21) —CH(CH₂SR^{5q})CO₂H,
(22) —CH(CH₂SH)NHSO₂NH₂,
(23) —CH(CH₂OH)CO₂H,
(24) —CH(CH₂OH)NHSO₂NH₂,
(25) —C(═O)CH₂CO₂H,
(26) —C(═O)(CH₂)₀₋₁CONH₂,
(27) —OSO₂NHR^{5q},
(28) —SO₂NHNH₂,
(29) —P(═O)(OH)₂, 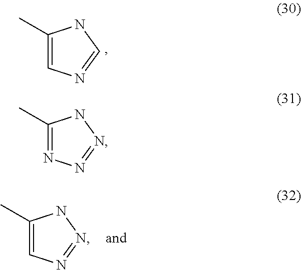
(33) —N(OH)C(═O)CR₁R₂,
wherein:
R₁ is selected from the group consisting of:
(1) —H,
(2) —OH,
(3) —OC₁-C₆-alkyl,
(4) —N(R^{2q},R^{3q}), and
(5) substituted or unsubstituted C₁-C₆-alkyl;
R₂ is selected from the group consisting of:
(1) H,
(2) substituted or unsubstituted C₁-C₆-alkyl,
(3) substituted or unsubstituted C₂-C₆-alkenyl,
(4) substituted or unsubstituted C₂-C₆-alkenyl,
(5) substituted or unsubstituted aryl,
(6) substituted or unsubstituted heterocyclyl, and
(7) substituted or unsubstituted heteroaryl,
or R₁ and R₂, together with the N atom to which they are attached can form a substituted or unsubstituted heterocyclic ring, having from 3 to 10 ring atoms, wherein 1-4 ring atoms of the heterocyclic ring are selected from N, O and S; and
each R^{1q}, R^{2q}, R^{3q}, R^{4q}, and R^{5q} is independently selected from the group consisting of H and C₁-C₆ alkyl.

7. The compound of claim 1, wherein the LpxC inhibitor is a compound of formula (I), or a stereoisomer, pharmaceutically acceptable salt, ester, or prodrug thereof: 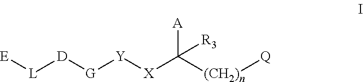
wherein
E is selected from the group consisting of:
(1) H,
(2) substituted or unsubstituted C₁-C₆-alkyl,
(3) substituted or unsubstituted C₂-C₆-alkenyl,
(4) substituted or unsubstituted C₂-C₆-alkynyl,
(5) substituted or unsubstituted C₃-C₁₀-cycloalkyl,
(6) substituted or unsubstituted aryl,
(7) substituted or unsubstituted heterocyclyl, and
(8) substituted or unsubstituted heteroaryl;
L is absent or selected from the group consisting of:
(1) substituted or unsubstituted C₁-C₆-alkyl,
(2) —(NR^{3L})₀₋₁—(CH₂)₀₋₄—NR^{3L}—(CH₂)₀₋₄—,
(3) —(NR^{3L})₀₋₁—C(R^{1L},R^{2L})—NR^{3L}—C(R^{1L},R^{2L})—,
(4) —C(R^{1L},R^{2L})—O—C(R^{1L},R^{2L})—,
(5) —(CH₂)₀₋₄—NR^{3L}—C(R^{1L},R^{2L})—CONH—(CH₂)₀₋₄—,
(6) —CO—C(R^{1L},R^{2L})—NHCO—,
(7) —CONR^{3L}—,
(8) —NR^{3L}CO—,
(9) —NR^{3L}—,
(10) —SO₂NR^{3L}—,
(11) —NR^{3L}—C(═O)—NR^{3L}—,
(12) substituted or unsubstituted C₃-C₁₀-cycloalkyl,
(13) substituted or unsubstituted aryl,
(14) substituted or unsubstituted heterocyclyl, and
(15) substituted or unsubstituted heteroaryl,
wherein:
each R^{1L}, R^{2L}, and R^{3L} is independently selected from the group consisting of:
(a) H,
(b) substituted or unsubstituted C₁-C₆-alkyl,
(c) C₁-C₆-alkyl substituted with aryl,
(d) C₁-C₆-alkyl substituted with heterocyclyl, and
(e) C₁-C₆-alkyl substituted with heteroaryl,
or R^{1L} and R^{3L}, together with the atoms to which they are attached can form a substituted or unsubstituted heterocyclic ring, having from 3 to 8 ring atoms, wherein 1-2 ring atoms of the heterocyclic ring are selected from N, O and S;
D is absent or selected from the group consisting of:
(1) substituted or unsubstituted C₃-C₁₀-cycloalkyl,
(2) substituted or unsubstituted aryl,
(3) substituted or unsubstituted heterocyclyl, and
(4) substituted or unsubstituted heteroaryl;
G is selected from the group consisting of:
(1) —(CH₂)₀₋₄—O—(CH₂)₀₋₄—,
(2) —(CH₂)₀₋₄—S—(CH₂)₀₋₄—,
(3) —(CH₂)₀₋₄—NR^{1G}—(CH₂)₀₋₄—,
(4) —C(═O)—,
(5) —NR^{1G}C(═O)—,
(6) —C(═O)NR^{1G}—,
(7) —(CH₂)₀₋₄NHCH₂C(═O)NR^{1G}—,
(8) —C≡C—,
(9) —C≡C—C≡C—,
(10) —CR^{2G}═CR^{2G}—,
(11) —S(═O)—,
(12) —SO₂—,
(13) —C(R^{3G})₂—S(═O)—,
(14) —S(═O)—C(R^{3G})₂—,
(15) —C(R^{3G})₂—SO₂—,
(16) —SO₂—C(R^{3G})₂—
(17) —CR^{3G}═CR^{3G}—CR^{3G}═CR^{3G}—,
(18) —C(R^{3G})₂—,
(19) —CR^{3G}═CR^{3G}—C≡C—,
(20) —C≡C—CR^{3G}═CR^{3G}—,
(21) —C(═O)—C≡C—,
(22) —C≡C—C(═O)—,
(23) substituted or unsubstituted C₃-C₁₀-cycloalkyl,
(24) substituted or unsubstituted aryl,
(25) substituted or unsubstituted heterocyclyl, and
(26) substituted or unsubstituted heteroaryl,
wherein:
R^{1G} is substituted or unsubstituted C₁-C₆-alkyl;
each R^{2G} and R^{3G} is independently selected from the group consisting of H, a halogen atom, and substituted or unsubstituted C₁-C₆-alkyl;
Y is absent or selected from the group consisting of:
(1) substituted or unsubstituted C₃-C₁₀-cycloalkyl,
(2) substituted or unsubstituted aryl,
(3) substituted or unsubstituted heterocyclyl, and
(4) substituted or unsubstituted heteroaryl;
X is selected from the group consisting of:
(1) —(C═O)NR₄—,
(2) —C₁-C₆-alkyl-(C═O)NR₄—,
(3) —C₂-C₆-alkenyl-(C═O)NR₄—,
(4) —C₂-C₆-alkynyl-(C═O)NR₄—,
(5) —CH₂NR₄—,
(6) —SO₂NR₄—,
(7) —S(═O)NR₄—,
(8) —NR₄C(═O)—, and
(9) —NR₄—,
or X and A, together with the atoms to which they are attached can form a heterocyclic ring, having from 5 to 8 ring atoms, wherein 1-2 ring atoms of the heterocyclic ring are selected from N, O and S,
or when Y is a bicyclic substituted or unsubstituted heterocyclyl or heteroaryl, then X is absent;
R₃ is H or substituted or unsubstituted C₁-C₆-alkyl, or R₃ and A, together with the atom to which they are attached can form a substituted or unsubstituted 3-10 membered cycloalkyl or a heterocyclic ring, having from 3 to 10 ring atoms, wherein 1-4 ring atoms of the heterocyclic ring are selected from N, O and S;
R₄ is (1) H or substituted or unsubstituted C₁-C₆-alkyl, or (2) R₄ and A, together with the atoms to which they are attached can form a substituted or unsubstituted heterocyclic ring, having from 3 to 8 ring atoms, wherein 1-2 ring atoms of the heterocyclic ring are selected from N, O and S, or (3) R₄ and Y, together with the atoms to which they are attached, form a bicyclic substituted or unsubstituted heterocyclyl or heteroaryl;
n is an integer from 0-6;
A is selected from the group consisting of:
(1) —C(R^{1a},R^{2a})OR^{3a},
(2) —C(R^{1a},R^{2a})N(R^{4a},R^{5a}),
(3) substituted or unsubstituted C₃-C₁₀-cycloalkyl,
(4) substituted or unsubstituted aryl,
(5) substituted or unsubstituted heterocyclyl, and
(6) substituted or unsubstituted heteroaryl,
wherein:
each R^{1a} and R^{2a} is independently selected from the group consisting of substituted or unsubstituted C₁-C₆-alkyl;
each R^{3a}, R^{4a}, and R^{5a} is independently selected from the group consisting of:
(a) H,
(b) a halogen atom,
(c) substituted or unsubstituted C₁-C₆-alkyl,
(d) substituted or unsubstituted aryl,
(e) substituted or unsubstituted heterocyclyl, and
(f) substituted or unsubstituted heteroaryl,
or R^{4a} and R^{5a} together with the N atom to which they are attached can form a substituted or unsubstituted heterocyclic ring, having from 5 to 8 ring atoms, wherein 1-2 ring atoms of the heterocyclic ring are selected from N, O and S; and
when A is —C(R^{1a},R^{2a})OR^{3a}, the compound is not 2-{[(4′-ethyl-1,1′-biphenyl-4-yl)carbonyl]amino}-3-hydroxy-3-methylbutanoic acid, 4′-ethyl-N-{2-hydroxy-1-[(hydroxyamino)carbonyl]-2-methylpropyl}-1,1′-biphenyl-4-carboxamide or N-{2-hydroxy-1-[(hydroxyamino)carbonyl]-2-methylpropyl}-4-(phenylethynyl)benzamide;
Q is absent or selected from the group consisting of:
(1) —C(═O)N(R₁,R₂),
(2) —NHC(═O)N(R₁,R₂),
(3) —N(OH)C(═O)N(R₁,R₂),
(4) —CH(OH)C(═O)N(R₁,R₂),
(5) —CH[N(R^{2q},R^{3q})]C(═O)N(R₁,R₂),
(6) —CHR^{1q}C(═O)N(R₁,R₂),
(7) —CO₂H,
(8) —C(═O)NHSO₂R^{4q},
(9) —SO₂NH₂,
(10) —N(OH)C(═O)R^{1q},
(11) —N(OH)SO₂R^{4q},
(12) —NHSO₂R^{4q},
(13) —SH,
(14) —CH(SH)(CH₂)₀₋₁C(═O)N(R₁,R₂),
(15) —CH(SH)(CH₂)₀₋₁CO₂H,
(16) —CH(OH)(CH₂)₀₋₁CO₂H,
(17) —CH(SH)CH₂CO₂R^{1q},
(18) —CH(OH)(CH₂)SO₂NH₂,
(19) —CH(CH₂SH)NHCOR^{1q},
(20) —CH(CH₂SH)NHSO₂R^{4q},
(21) —CH(CH₂SR^{5q})CO₂H,
(22) —CH(CH₂SH)NHSO₂NH₂,
(23) —CH(CH₂OH)CO₂H,
(24) —CH(CH₂OH)NHSO₂NH₂,
(25) —C(═O)CH₂CO₂H,
(26) —C(═O)(CH₂)₀₋₁CONH₂,
(27) —OSO₂NHR^{5q},
(28) —SO₂NHNH₂,
(29) —P(═O)(OH)₂, 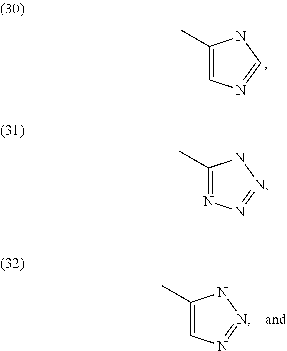
(33) —N(OH)C(═O)CR₁R₂,
wherein:
R₁ is selected from the group consisting of:
(1) —H,
(2) —OH,
(3) —OC₁-C₆-alkyl,
(4) —N(R^{2q},R^{3q}), and
(5) substituted or unsubstituted C₁-C₆-alkyl;
R₂ is selected from the group consisting of:
(1) H,
(2) substituted or unsubstituted C₁-C₆-alkyl,
(3) substituted or unsubstituted C₂-C₆-alkenyl,
(4) substituted or unsubstituted C₂-C₆-alkenyl,
(5) substituted or unsubstituted aryl,
(6) substituted or unsubstituted heterocyclyl, and
(7) substituted or unsubstituted heteroaryl,
or R₁ and R₂, together with the N atom to which they are attached can form a substituted or unsubstituted heterocyclic ring, having from 3 to 10 ring atoms, wherein 1-4 ring atoms of the heterocyclic ring are selected from N, O and S; and
each R^{1q}, R^{2q}, R^{3q}, R^{4q}, and R^{5q} is independently selected from the group consisting of H and C₁-C₆ alkyl.

8. The pharmaceutical composition of claim 1, wherein the LpxC inhibitor is selected from the group consisting of (R)—N-hydroxy-2-(4-methoxyphenyl)-4,5-dihydrooxazole-4-carboxamide (LpxCi-1); (S)-2-(3,4-dimethoxy-5-propylphenyl)-N-hydroxy-4,5-dihydrooxazole-4-carboxamide (LpxCi-2); N-((2S,3R)-3-hydroxy-1-(hydroxyamino)-1-oxobutan-2-yl)-4-((4-(morpholinomethyl)phenyl)ethynyl)benzamide (LpxCi-3); (S)—N-(3-amino-1-(hydroxyamino)-3-methyl-1-oxobutan-2-yl)-4-(cyclopropylbuta-1,3-diynyl)benzamide (LpxCi-4); (S,E)-N-(3-amino-1-(hydroxyamino)-3-methyl-1-oxobutan-2-yl)-4-(4-cyclopropylbut-3-en-1-ynyl)benzamide (LpxCi-5); and (S)-4-(cyclopropylbuta-1,3-diynyl)-N-(3-hydroxy-1-(hydroxyamino)-3-methyl-1-oxobutan-2-yl)benzamide (LpxCi-6).

9. The pharmaceutical composition of claim 1, wherein the LpxC inhibitor is a compound having formula II-A, II-B or II-C, or a stereoisomer, pharmaceutically acceptable salt, ester, or prodrug thereof: 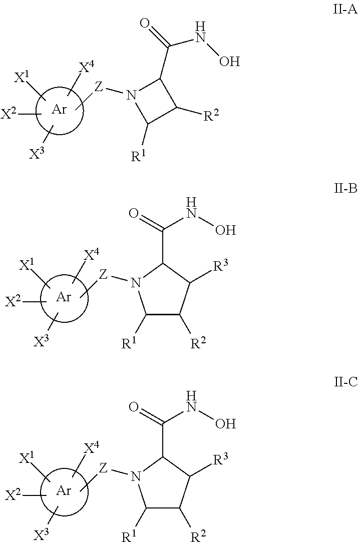
wherein:
X¹, X², X³, and X⁴ are independently selected from the group consisting of hydrogen, alkyl, haloalkyl, alkylthio, alkylsulfinyl, alkylsulfonyl, hydroxyalkyl, alkoxy, alkoxyalkyl, haloalkoxy, alkenyl, alkenoxy, alkenoxyalkyl, alkynyl, alkynyloxy, nitro, halo, hydroxy, cycloalkyl, cycloalkylalkyl, arylalkoxy, arylalkoxyalkyl, haloalkylthio, haloalkylsulfinyl, haloalkylsulfonyl, haloarylalkyl, haloarylalkynyl, alkylsilylalkynyl, aryl, alkynyloxy, anaminocarbonylalkyl, carboxylate, carboxyl, carboxamide, heterocycle, and substituted heterocycle;
R¹ and R³ are independently selected from the group consisting of hydrogen, alkyl, haloalkyl, hydroxyalkyl, alkenyl, alkynyl, cycloalkyl, aryl, substituted aryl, heteroaryl, substituted heteroaryl, halo, hydroxy, alkoxy, and —O—R4 where R4 is a substituted or unsubstituted aryl;
R² is selected from the group consisting of hydrogen, alkyl, haloalkyl, hydroxyalkyl, alkenyl, alkynyl, cycloalkyl, aryl, substituted aryl, heteroaryl, substituted heteroaryl, halo, hydroxy, alkoxy, and —O—R⁴ where R⁴ is a substituted or unsubstituted aryl; and
Z is —CH²— or —C(O)—.

10. The pharmaceutical composition of claim 1, wherein the LpxC inhibitor is a compound having formula III, or a stereoisomer, pharmaceutically acceptable salt, ester, or prodrug thereof: 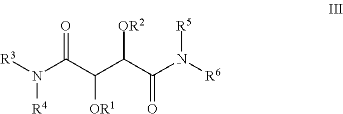
wherein:
(i) each of R¹ and R² independently is hydrogen or alkyl;
(ii) R³ and R⁴ taken together with the nitrogen to which they shown attached is heterocyclyl or heteroaryl, said heterocyclyl or heteroaryl having 1-3 heteroatoms including said nitrogen, said heterocyclyl or heteroaryl being optionally fused with aryl, heteroaryl, cycloalkyl, or heterocyclyl; wherein said heterocyclyl or heteroaryl comprising R³ and R⁴ is substituted with one or two substituents, each substituent being independently selected from the group consisting of aryl and alkynyl; wherein said aryl substituent is unsubstituted or is optionally substituted with one or two moieties selected independently from the group consisting of perhaloalkyl, halo, alkyl, alkoxy, cyano, perhaloalkoxy, and alkynyl moiety, wherein said alkynyl moiety is substituted with an aryl radical; wherein said alkynyl substituent is substituted with an aryl moiety, wherein said aryl moiety is unsubstituted or optionally substituted with one to three radicals selected from the group consisting of perhaloalkyl, halo, alkyl, alkoxy, cyano, and perhaloalkoxy; and
(iii) each of R⁵ and R⁶ is alkyl, or alternatively R⁵ and R⁶ taken together with the nitrogen to which they shown attached is heterocyclyl having 1-3 heteroatoms including said nitrogen; wherein said heterocyclyl comprising R⁵ and R⁶ is unsubstituted or optionally substituted with an aryl substituent; wherein said aryl substituent is unsubstituted or optionally substituted with one to three moieties independently selected from the group consisting of perhaloalkyl, halo, alkyl, alkoxy, cyano, and perhaloalkoxy; with the proviso that the aryl substituent of said heterocyclyl or heteroaryl comprising R³ and R⁴ can be unsubstituted or optionally independently substituted with one to three moieties independently selected from the group consisting perhaloalkyl, halo, alkyl, alkoxy, cyano, and perhaloalkoxy only when R⁵ and R⁶ taken together with the nitrogen to which R⁵ and R⁶ are attached is heterocyclyl.

11. A method for treating a patient with a gram-negative bacterial infection, comprising co-administering a synergistic amount, preferably an in vivo synergistic amount, of an antibacterial agent and an inhibitor of LpxC.

12. The method of claim 11, wherein the antibacterial agent is selected from the group consisting of vancomycin, linezolid, azithromycin, imipenem, teicoplanin, daptomycin, clindamycin, rifampin, cefotaxime, gentamicin, novobiocin, and telavancin.

13. The method of claim 12, wherein the antibacterial agent is vancomycin or rifampin.

14. The method of claim 11, wherein the LpxC inhibitor is a compound of formula I, II-A, II-B, II-C, or III, or a stereoisomer, pharmaceutically acceptable salt, ester, or prodrug thereof.

15. A method of suppressing the emergence of resistance to an antibacterial agent, said method comprising co-administering a synergistic amount of an antibacterial agent and an inhibitor of LpxC.

16. The method of claim 15, wherein the synergistic amount of the antibacterial agent and the inhibitor of LpxC is an in vivo synergistic amount.

17. The method of claim 15, wherein the antibacterial agent is selected from the group consisting of vancomycin, linezolid, azithromycin, imipenem, teicoplanin, daptomycin, clindamycin, rifampin, cefotaxime, gentamicin, novobiocin, and telavancin.

18. The method of claim 17, wherein the antibacterial agent is vancomycin or rifampin.

19. The method of claim 15, wherein the LpxC inhibitor is a compound of formula I, II-A, II-B, II-C, or III, or a stereoisomer, pharmaceutically acceptable salt, ester, or prodrug thereof.
